(19) United States
(12) Patent Application Publication
(10) Pub. No.: US 2006/0089340 A1
(43) Pub. Date: Apr. 27, 2006

(54) Novel steroids and steroid mimetics; method for their production; and use thereof

(76) Inventors: Helmut Kasch, Jena (DE); Beatrice Liedtke, Jena (DE)

Correspondence Address: FROMMER LAWRENCE & HAUG, 745 FIFTH AVENUE- 10TH FL., NEW YORK, NY, 10151, US

(21) Appl. No.: 11/190,700

(22) Filed: Jul. 27, 2005

Related U.S. Application Data
(63) Continuation-in-part of application No. PCT/DE04/00152, filed on Jan. 28, 2004.

(30) Foreign Application Priority Data
Jan. 28, 2003 (DE) 103 03 667.9
Jan. 28, 2003 (DE) 103 03 671.7
Jan. 28, 2003 (DE) 103 03 668.7
Jan. 28, 2003 (DE) 103 03 670.9

(51) Int. Cl. A61K 31/56; C07J 1/00

(52) U.S. Cl. 514/178; 552/617

(57) ABSTRACT

The invention relates to the production and use of novel 14β-alkyl-18-norsteroids, 8α,14β-dialkyl-18-norsteroids, spiro[cyclopentano-perhydronaphthalin]-3,1′-pentanes or hexanes in addition to 14β-fluoro-15β,17β-dihydroxy-steroids, by reacting 17-hydroxy-14,15-epoxi-steroids with water-free acids or Lewis acids and followed by derivatisation. The resulting compounds can be used as intermediate products for or as medicaments in human and veterinary medicine, whereby the emphasis is on improving inflammatory processes, hormone replacement therapy and the use thereof as enzyme inhibitors in the treatment of tumours.

### REFERENCE TO RELATED APPLICATIONS

This application is a continuation-in-part of PCT/DE2004/000152 filed Jan. 28, 2004 and published as WO 2004/067547 on Aug. 12, 2004, which claims priority from German patent application numbers 103 03 667.9, 103 03 671.7, 103 03 668.7, and 103 03 670.9, all of which were filed on Jan. 28, 2003.

Each of the above referenced applications, and each document cited in this text (“application cited documents”) and each document cited or referenced in each of the application cited documents, and any manufacturer's specifications or instructions for any products mentioned in this text and in any document incorporated into this text, are hereby incorporated herein by reference; and, technology in each of the documents incorporated herein by reference can be used in the practice of this invention.

It is noted that in this disclosure, terms such as “comprises”, “comprised”, “comprising”, “contains”, “containing” and the like can have the meaning attributed to them in U.S. Patent law; e.g., they can mean “includes”, “included”, “including” and the like. Terms such as “consisting essentially of” and “consists essentially of” have the meaning attributed to them in U.S. Patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. Patent law; namely, that these terms are closed ended.

### SUMMARY OF THE INVENTION

The invention concerns novel 14β-alkyl-18-norsteroids with general formula I: 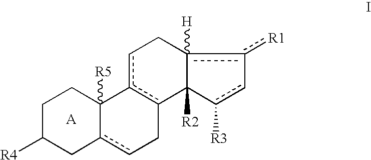
wherein
R₁=O, α- or β-OH, H, oxyalkyloxy (alkyl containing 2 to 5 C atoms), 17,2′-spirooxirane or of the type 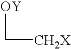
in which Y=H, alkyl (containing 1 to 6 C atoms), alkanoyl (containing 1 to 12 C atoms) and X=halogen such as chorine, bromine, iodine or fluorine, pseudohalogen such as N₃, SCN, SeCN, CN, or alkyloxy (containing 1 to 6 C atoms) and alkanoyloxy (alkyl containing 1 to 12 C atoms), OSO₂NR₆R₇ with R₆ and R₇=H, alkyl, cycloalkyl (containing 1 to 6 C atoms) or cycloalkyl (containing 1 to 7 C atoms), or alkanoyl (alkyl containing 1 to 8 C atoms);
R₂=methyl or ethyl;
R₃=O, OH, H, OSO₂NR₆R₇ with R₆ and R₇=H, alkyl, cycloalkyl (containing 1 to 6 C atoms) or cycloalkyl (containing 1 to 7 C atoms), alkanoyl (alkyl containing 1 to 8 C atoms);
R₄=H, O, OH, O-alkyl containing 1 to 12 C atoms, alkyloxymethyloxy (alkyl containing 1 to 3 C atoms), alkanoyloxy (containing 1 to 12 C atoms), aralkanoyloxy (alkyl containing 1 to 4 C atoms) or aralkyloxy (alkyl containing 1 to 4 C atoms), OSO₂N₇ with R₆ and R₇=H, alkyl, cycloalkyl (containing 1 to 6 C atoms) or cycloalkyl (containing 1 to 7 C atoms), alkanoyl (alkyl containing 1 to 8 C atoms), —OSO₂OR with R=H, alkyl or cycloalkyl (alkyl containing 1 to 7 C atoms);
R₅=α or β H, methyl, chlorine or a methylene group bridging after C5 in the case in which A does not designate an aromatic ring or there is no double bond between C atoms 5 and 10;
A=an aromatic ring or a partially or fully hydrogenated ring;
and additional double bonds may be present between C atoms 4 and 5; 5 and 6; 9 and 11; 8 and 9; 13 and 17 as well as 15 and 16;
as well as salts and esters with acids such as sulphuric acid, sulphamic acid, N-substituted sulphamic acid or phosphoric acids;
novel 8α,14β-dialkyl-18-norsteroids with general formula I: 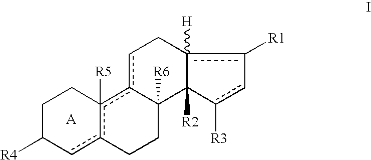
wherein
R₁=O, α- or β-OH, H, 17-spiro-2′-oxirane or of the type 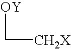
in which Y=H, alkyl (containing 1 to 6 C atoms), alkanoyl (containing 1 to 12 C atoms) and X=halogen such as chorine, bromine, iodine or fluorine, pseudohalogen such as N₃, SCN, SeCN, CN, or alkyloxy (containing 1 to 6 C atoms) and alkanoyloxy (alkyl containing 1 to 12 C atoms), alkyloxy (alkyl containing 1 to 12 C atoms), alkyloxymethyloxy (alkyl containing 1 to 3 C atoms), alkanoyloxy (alkyl containing 1 to 12 C atoms), aralkyloxy (alkyl containing 1 to 4 C atoms), oxyalkyloxy (alkyl containing 2 to 6 C atoms), S—CH₂—CH₂S, O—CH₂—CH₂S, trialkylsilyloxy (alkyl containing 1 to 4 C atoms), OSO₂NR₆R₇ with R₆ and R₇=H, alkyl (containing 1 to 6 C atoms) or cycloalkyl (containing 1 to 7 C atoms), or alkanoyl (alkyl containing 1 to 8 C atoms);
R₂=methyl or ethyl;
R₃=H, O, OH, alkyloxy (alkyl containing 1 to 12 C atoms), alkyloxymethyloxy (alkyl containing 1 to 3 C atoms), alkanoyloxy (alkyl containing 1 to 12 C atoms), aralkanoyloxy (alkyl containing 1 to 4 C atoms) or aralkyloxy (alkyl containing 1 to 4 C atoms), OSO₂NR₆R₇ with R₆ and R₇=H, alkyl (containing 1 to 6 C atoms) or cycloalkyl (containing 1 to 7 C atoms) alkanoyl (alkyl containing 1 to 8 C atoms);
R₄=H, O, alkyloxy (alkyl containing 1 to 12 C atoms), alkyloxymethyloxy (alkyl containing 1 to 3 C atoms), aralkyloxy (alkyl containing 1 to 4 C atoms), alkanoyloxy (containing 1 to 12 C atoms), aralkanoyloxy (alkyl containing 1 to 4 C atoms) OSO₂NR₆R₇ with R₆ and R₇=H, alkyl (containing 1 to 6 C atoms) or cycloalkyl (containing 1 to 7 C atoms), alkanoyl (alkyl containing 1 to 8 C atoms);
R₅=α or β H, chlorine or a methylene group bridging after C5 in the case in which A does not designate an aromatic ring or there is no double bond between C atoms 5 and 10;
R₆=α-methyl, α-ethyl or α-methyoxymethyl;
A=an aromatic ring or a partially or fully hydrogenated ring;
and additional double bonds may be present between C atoms 4 and 5, 5 and 10, 9 and 10, 9 and 11, 13 and 17, 15 and 16, and/or 16 and 17;
as well as salts with amines and esters with acids such as sulphuric acid, sulphamic acid, N-substituted sulphamic acid or phosphoric acids;
novel spiro[cyclopentano-perhydronaphthalene]-3,1′-pentanes (-hexanes) with general formula I: 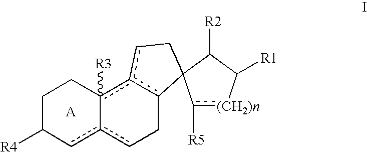
wherein
R₁=H, OH, O, O-alkyl (alkyl containing 1 to 12 C atoms), alkanoyloxy (alkyl containing 1 to 12 C atoms), alkyloxymethyloxy (alkyl containing 1 to 3 C atoms), aralkanoyloxy (alkyl containing 1 to 4 C atoms), oxyalkyloxy (alkyl containing 2 to 5 C atoms), S—CH₂—CH₂—S, O—CH₂—CH₂—S, 17-spiro-2′-oxirane of the 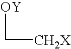
type with Y=H, alkyl (containing 1 to 6 C atoms), alkanoyl (alkyl containing 1 to 12 C atoms) and X=H, halogen such as chlorine, bromine, iodine or fluorine, pseudohalogen such as N₃, SCN, SeCN, CN or alkyloxy (containing 1 to 6 C atoms) and alkanoyloxy (alkyl containing 1 to 12 C atoms), OSO₂NR₆R₇ with R₆ and R₇=H, alkyl (containing 1 to 6 C atoms) or cycloalkyl (containing 1 to 7 C atoms) or alkanoyl (alkyl containing 1 to 8 C atoms);
R₂=methyl or ethyl and H;
R₃=methyl or H in the case in which A does not designate an aromatic ring and there is no double bond between C atoms 5 and 10 or 10 and 9;
R₄=H, OH, O, oxyalkyloxy (alkyl containing 2 to 5 C atoms), alkyloxy (alkyl containing 1 to 12 C atoms), alkyloxymethyloxy (alkyl containing 1 to 3 C atoms), alkanoyloxy (alkyl containing 1 to 12 C atoms), aralkyloxy (alkyl containing 1 to 3 C atoms), aralkanoyloxy (alkyl containing 1 to 4 C atoms), OSO₂NR₆R₇ with R₆ and R₇=H, alkyl (containing 1 to 6 C atoms) or cycloalkyl (containing 1 to 7 C atoms), alkanoyl (alkyl containing 1 to 8 C atoms);
R₅=H, OH, O, alkyloxy (alkyl containing 1 to 12 C atoms), alkoxymethyloxy (alkyl containing 1 to 3 C atoms), alkanoyloxy (alkyl containing 1 to 12 C atoms), aralkanoyloxy (alkyl containing 1 to 4 C atoms), OSO₂NR₆R₇ with R₆ and R₇=H, alkyl (containing 1 to 6 C atoms) or cycloalkyl (containing 1 to 7 C atoms), alkanoyl (alkyl containing 1 to 8 C atoms);
A=an aromatic ring or a partially or fully hydrogenated ring;
n=1 or 2;
and additional double bonds may be present between C atoms 4 and 5; 5 and 6, 8 and 9, 9 and 10, 5 and 10, 9 and 11, and/or 15 and 16;
as well as salts with amines and esters with acids such as sulphuric acid, sulphamic acid, N-substituted sulphamic acid or phosphoric acids; and

The invention concerns novel 14β-fluoro-15β,17β-dihydroxy-steroids with general formula I: 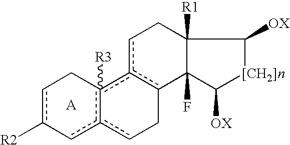
wherein
R₁=methyl or ethyl;
R₂=O, OH, O-alkyl (alkyl containing 1 to 12 C atoms), alkyloxymethyloxy (alkyl containing 1 to 3 C atoms), alkanoyloxy (alkyl containing 1 to 12 C atoms), aralkanoyloxy (alkyl containing 1 to 4 C atoms) or aralkyloxy (alkyl containing 1 to 4 C atoms), OSO₂NR₆R₇ with R₆ and R₇=H, alkyl (containing 1 to 6 C atoms) or cycloalkyl (containing 1 to 7 C atoms), alkanoyl (alkyl containing 1 to 8 C atoms), OSO₃H, OSO₃—, OSO₂R [in which R=alkyl or cycloalkyl residue (alkyl containing 1 to 7 C atoms)];
R₃=methyl or H in the case in which A does not designate an aromatic ring or there is no double bond between C atoms 5 and 10 as well as 9 and 10;
X=H, SO₂NR₆R₇ [in which R₆ and R₇=H, alkyl (containing 1 to 6 C atoms) or cycloalkyl (containing 1 to 7 C atoms), alkanoyl (alkyl containing 1 to 8 C atoms)], alkyl (alkyl containing 1 to 12 C atoms), alkyloxymethyl (alkyl containing 1 to 3 C atoms), alkanoyl with 1 to 12 C atoms, aralkanoyl (alkyl containing 1 to 4 C atoms) or aralkyl (alkyl containing 1 to 4 C atoms);
n=1 or 2;
A=an aromatic ring or a partially or fully hydrogenated ring;
and additional double bonds may be present between C atoms 2 and 3, 4 and 5; 5 and 6; 5 and 10, 9 and 10, 9 and 11; and 8 and 9;
as well as salts with amines and esters with acids such as sulphuric acid, sulphamic acid, N-substituted sulphamic acid or phosphoric acids.

The term “aryl” in residues such as aralkanoyloxy and the like means phenyl or alkylphenyl (alkyl containing 1 to 4 C atoms) in particular.

N-substituted sulphamic acids carry mono- or dialkyl substituents (alkyl containing 1 to 6 C atoms) or alkanoyl substituents (alkyl containing 1 to 8 C atoms).

Methods for preparing the novel compounds and also for the preparation of their pharmaceutical formulations are also described.

The starting materials 17β-hydroxy-14α,15α-epoxide (K Ponsold et al, J Prakt Chem 1981, 323, 819; G Schubert et al, Pharmaz 1979, 34, 323) and 17α-hydroxy-14β,15β-epoxide are known from the prior art. Finally, 17-keto-Δ¹⁴ steroids are converted into 17-keto-14β,15β epoxides using peracids and are reduced to the 17α-alcohol with DIBAH by selective reduction (H Künzer et al, Tet Lett 1995, 36, 1237).

Further, 17β-hydroxy-14β,15β-epoxides are known from the prior art (H Kasch, Tet Lett 1996, 37, 8349). They are produced from 17β-hydroxy-Δ¹⁴ steroids by epoxidation with peracids or by Sharpless epoxidation or using chloral hydrate/H₂O₂ from those olefins.

Further, the starting materials, Wiechert ketone and the alkylization synthones for the preparation of 8α,14β-dialkyl-18-norsteroids are known from the prior art. The present compounds are specifically modified structures which in their broadest sense can be categorized as 19-norsteroids or 19-norfusidans.

The compounds of the invention and the methods for their preparation are novel; their biological effectiveness has not previously been described.

The aim of the invention it to provide novel 14β-alkyl-18-norsteroids, 8α,14β-dialkyl-18-norsteroids, spiro[cyclopentano-perhydronaphthalene]-3,1′-pentanenes (hexanenes) and 14β-fluoro, 15β-hydroxysteroids and to disclose a method for their preparation, which have different biological and structural profiles with respect to conventional steroid hormones, and which aim to reduce systemic effects.

The invention provides novel 14β-alkyl-18-norsteroids as defined in claims 1 to 4.

Examples of preferred compounds within the context of the invention are as follows:
15α-hydroxy-3-methoxy-17-oxo-14β-methyl-13α-H-gona-1,3,5(10)-triene;
15α-hydroxy-3-methoxy-17-oxo-14β-methyl-13-H-gona-1,3,5(10)-triene;
3,17β-dihydroxy-14β-methyl-13α-H-gona-1,3,5(10)-triene;
3,17β-disulphamoyloxy-14β-methyl-13α-H-gona-1,3,5(10)-triene;
3-hydroxy-14β-methyl-3-sulphamoyloxy-13α-H-gona-1,3,5(10)-triene;
17β-hydroxy-14β-methyl-3-sulphamoyloxy-13α-H-gona-1,3,5(10)-triene;
3-methoxy-14β-methyl-17-oxo-15α-sulphamoyloxy-13α-H-gona-1,3,5(110)-triene;
14β-methyl-17-oxo-3-sulphamoyloxy-13α-H-gona-1,3,5(110)-triene;
14β-methyl-3,17β,14α-trisulphamoyloxy-13α-H-gona-1,3,5(110)-triene;
N-[15α-methyleneoxycarbonyloxy-3-methoxy-15β-methyl-17-oxo-13β-H-oestra-1,3,5(10)-triene]-N,N,N-triethylammonium iodide;
15α,17′-dihydroxy-14′-methyl-18-nor-13α-H-oestra-4-en-3-one;
15α,17β-disulphamoyloxy-14′-methyl-18-nor-13α-H-oestra-4-en-3-one;
15α-hydroxy-3-methoxy-17-oxo-8α,14′-dimethyl-13α-H-gona-1,3,5(10),9(11)-tetraene;
3-methoxy-17-oxo-8α,14β-dimethyl-13α-H-gona-1,3,5(10),9(11),15-pentaene;
17′-hydroxy-3-methoxy-8α,14′-dimethyl-13α-H-gona-1,3,5(10)-triene;
17α-hydroxy-3-methoxy-8α,14′-dimethyl-13α-H-gona-1,3,5(10)-triene;
3,17β-dihydroxy-8α,14′-dimethyl-13α-H-gona-1,3,5(10)-triene;
3,17β-dihydroxy-8α,14β-dimethyl-13′-H-gona-1,3,5(10)-triene;
3,17′-dihydroxy-8α,14′-dimethyl-13α-H-gona-1,3,5(10),9(11)-tetraene;
3,17′-disulphamoyloxy-8α,14β-dimethyl-13′-H-gona-1,3,5(10)-triene;
N-[17α-methyleneoxycarbonyloxy-3-methoxy-8α,14′-dimethyl-13α-H-gona-1,3,5(10)-triene],
N,N,N-triethylammonium iodide;
spiro-[2,3,3a,4,5,9b-hexahydro-7-methoxy-1H-cyclopenta[a]naphthalene]-3,1′-[2′-S-methyl, 3′-oxo-5′-R-hydroxyl-pentane];
spiro-[2,3,3a,4,5,9b-hexahydro-7-methoxy-1H-cyclopenta[a]naphthalene]-3,1′-[2′-S-methyl, 3′-oxo-]-pentene];
spiro-[2,3,3a,4,5,9b-hexahydro-7-methoxy-1H-cyclopenta[a]naphthalene]-3,1′-[2′-S-methyl, 3′-oxo-5′-R-sulphamoyloxy]-pentane];
spiro-[2,3,3a,4,5,9b-hexahydro-7-methoxy-1H-cyclopenta[a]naphthalene]-3,1′-[2′-S-methyl, 3′-oxo-]-pentene];
spiro-[2,3,3a,4,5,9b-hexahydro-7-sulphamoyloxy-1H-cyclopenta[a]naphthalene]-3,1′-[2′-S-methyl, 3′-oxo-]-pentene];
spiro-[2,3,3a,4,5,9b-hexahydro-7-sulphamoyloxy-1H-cyclopenta[a]naphthalene]-3,1′-[2′-S-methyl, 3′-oxo-5′-R-sulphamoyloxy]-pentane];
N-{spiro-[2,3,3a,4,5,9b-hexahydro-7-methoxy-1H-cyclopenta[a]naphthalene]-3,1′-[2′-S-methyl,
3′-oxo-5′-methyleneoxycarbonyloxy]-pentane}, N,N,N-triethylammonium iodide;
14β-fluoro-15β,17-dihydroxy-3-methoxy-13β-methyl-gona-1,3,5(10)-triene;
14β-fluoro-3,15β,17β-trihydroxy-13β-methyl-gona-1,3,5(10)-triene;
14β-fluoro-15 β,17β-dihydroxy-19-nor-androsten-3-one;
14β-fluoro-3-methoxy-15,17β-disulphamoyloxy-13β-methyl-gona-1,3,5(10)-triene;
14β-fluoro-15β,17β-dihydroxy-3-sulphamoyloxy-13β-methyl-gona-1,3,5(10)-triene;
N-[17β-methyleneoxycarbonyloxy-3-methoxy-13β-methyl-gona-1,3,5(10)-triene]-N,N,N-triethylammoninum iodide.

The present invention also concerns pharmaceutical compositions containing as active principle at least one 14β-alkyl-18-norsteroid, a 8α,14β-dialkyl-18-norsteroid, a spiro[cyclopentanoperhydronaphthalene]-3,1′-pentane (hexane) or 14β-fluoro,15β-hydroxysteroid with the corresponding general formula I, wherein said compositions may comprise suitable excipients, carriers and additives and/or stabilizers.

The compounds of the invention, which contain alkyl groups in the 14 and/or 8 positions, have a modified profile compared with conventional oestrogens and androgens, and do not suffer from undesirable systemic side effects. This means, inter alia, that they can also be used as carriers and active ingredients for other pharmaceutical uses. The structures of the invention are more suitable, for example, than cholesterol or sitosteroid derivatives as regards carrier properties. Further, structures derived from the 14β-fluoro-15β-hydroxysteroids and the spirocyclopentanes can be used in hormone replacement therapy (HRT) because of their specific agonistic and antagonistic partial effects. The compounds of the invention may also be used to strengthen and stimulate host rejection, as radical scavengers and as enzyme inhibitors. Thus, diseases such as Alzheimer's, tumours or infections may be treated, as well as general conditions, and the retention of physical and mental mobility in the aged, can be improved. Further, the pharmaceutical compositions of the compounds of the invention may be used in combination with other active ingredients, for example steroid sulphamates, to increase effectiveness.

The pharmaceuticals of the invention are produced with the usual solid or liquid diluents and the usual pharmaceutical excipients depending on the desired administration route, in a suitable dose, in known manner. Preferred compositions are in administration forms for oral administration in the form of tablets, dragees, pills, powders and slow-release forms. The compounds of the invention can also be used in suspensions or solutions for use in transdermal systems. The compounds of the invention may be used as scavengers, as steroid hydrogenase, carboanhydrase and/or sulphatase inhibitors and may be used in treating infections because of their antibacterial and antimycotic effects. Further, they can be used as intermediates for the synthesis of further pharmacologically effective compounds.

For the preparation of 14β-alkyl-18-norsteroids, 17β-hydroxy-14α,15α-epoxy steroids with general formula II: 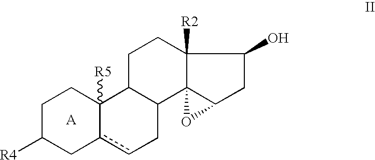
in which R₂=methyl or ethyl, R₄=H, O, OH, O-alkyl containing 1 to 12 C atoms, alkyloxymethyloxy (alkyl containing 1 to 3 C atoms), alkanoyloxy containing 1 to 12 C atoms, aralkanoyloxy (alkyl containing 1 to 4 C atoms) or aralkyloxy (alkyl containing 1 to 4 C atoms), R₅=methyl or H in the case in which A does not designate an aromatic ring, A=an aromatic ring, a partially hydrogenated or fully hydrogenated ring and may contain additional double bonds between C atoms 4 and 5; 5 and 6,
are converted, in an aprotic solvent such as toluene in the presence of an organic acid such as p-toluenesulphonic acid, methanesulphonic acid, trifluoromethanesulphonic acid, H₂SO₄*SO₃, HSO₃F, HSO₃Cl, HB(HSO₄)₄, HSbF₆ or a Lewis acid such as boron trifluoride diethyl ether complex, boron tribromide, boron trichloride, ZnCl_{2,} SbF₅ or Sbl5, with heating, to thereby isolate 14β-alkyl-18-norsteroids with general formula I 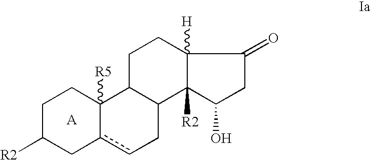
in which R₂, R₄, R₅ and A have the meanings described above and may contain additional double bonds between C atoms 4 and 5; 5 and 6;
for the production of the compounds of the invention of the 8α,14β-dialkyl-18-norsteroid type, the Weichert ketone (II) is pre-treated in an ether such as dimethoxyethane or tetrahydrofuran with a base such as sodium hydride or potassium tert-butylate and is reacted with an alkylation synthone (III) such as 2(3-alkyloxyphenyl)-tosylate or -bromide and is transformed, after further action with a base using an alkylation agent such as methyl, ethyl or methoxymethyl iodide, into 17β-tert-butoxy-8α-alkyl-9-oxo-9,10-seco-gona-1,3,5(10),14-tetraene (IV): 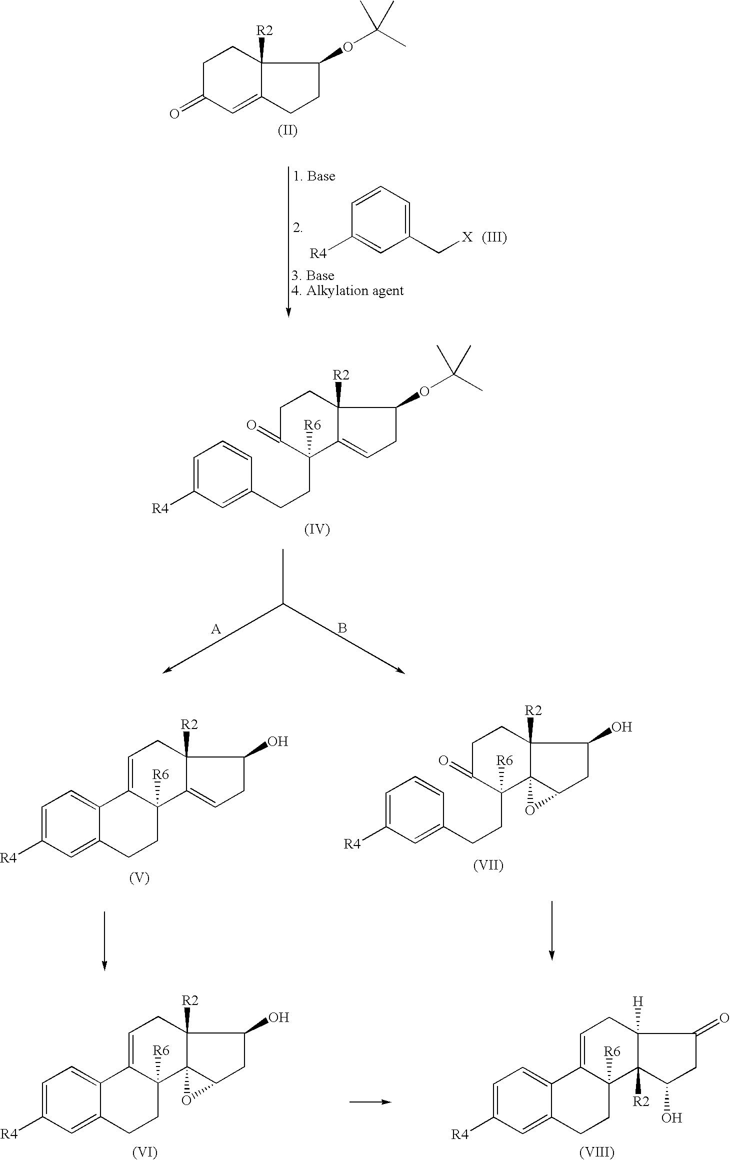
in which R₂=methyl or ethyl, R₄=alkyloxy (alkyl containing 1 to 12 C atoms), alkyloxymethyloxy (alkyl containing 1 to 3 C atoms), R₆=methyl, ethyl or alkyloxymethyloxy (alkyl containing 1 to 3 C atoms), X=tosyloxy, bromide, chloride or iodide;
and this is isolated either via route A:
transformation by aldol condensation using an organic acid such as p-toluenesulphonic acid or a Lewis acid such as boron trifluoride diethyl ether complex in an aprotic solvent such as toluene, benzene, a halogenated hydrocarbon or tetrahydrofuran into 17β-hydroxy-8α-alkyl-gona-1,3,5(10),9(11),14-pentaene with general formula (V), in which R₄ and R₂ have the meanings given above, the latter being reduced, by selective epoxidation using peracids such as m-chloroperbenzoic acid or by Sharpless epoxidation using vanadyl acetyl acetonate and tert-butyl hydroperoxide in any solvent such as methylene chloride or toluene, to the 14α,15α-epoxide with general formula (VI), the latter being transformed, by treatment with a Lewis acid such as boron trifluoride diethyl ether complex in a suitable anhydrous solvent such as toluene, benzene, a halogenated hydrocarbon or tetrahydrofuran, into 8α,14β-dialkyl-15α-hydroxy-17-oxo-gona-1,3,5(10),9(11)-tetraene (I or VIII with the meanings given above for R₂ and R₄);
or via route B:
reduction to the seco-14α,15α-epoxide (VII) by selective epoxidation using peracids which, by means of an organic acid or Lewis acid such as boron trifluoride diethyl ether complex in a suitable anhydrous solvent such as toluene, is transformed into 8α,14β-dialkyl-15α-hydroxy-17-oxo-gona-1,3,5(10),9(11)-tetraene (I or VIII with the meanings given above for R₂ and R₄);
for the production of compounds of the invention of the spiro[cyclopentanoperhydronaphthalene]-3,1′-pentanes (-hexanes) type, 17α-hydroxy-14β,15β-epoxysteroids with general formula II: 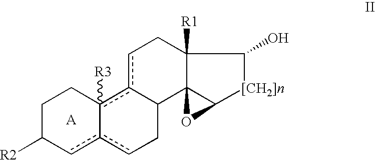
in which R₁=methyl or ethyl, R₂=OH, O, alkoxy (alkyl containing 1 to 12 C atoms), alkyloxymethyloxy (alkyl containing 1 to 3 C atoms), alkanoyloxy (alkyl containing 1 to 12 C atoms), aralkanoyloxy (alkyl containing 1 to 4 C atoms), aralkyloxy (alkyl containing 1 to 3 C atoms), aralkanoyloxy (alkyl containing 1 to 4 C atoms), R₃=methyl or H in the case in which A does not designate an aromatic ring and there is no double bond between C atoms 5 and 10 and 9 and 10; n=1 or 2; A=an aromatic, partially or completely hydrogenated ring and additional double bonds may be present between C atoms: 4 and 5, 5 and 6, 5 and 10, 9 and 10 and/or 9 and 11, are converted, in an anhydrous aprotic solvent such as toluene, benzene, heptane, an ether, a halogenated hydrocarbon or mixtures of solvents in the presence of an acid such as p-toluenesulphonic acid, methane sulphonic acid, trifluoromethanesulphonic acid, H₂SO₄*SO₃, HSO₃F, HSO₃Cl, HB(HSO₄)₄, HSbF₆ or a Lewis acid such as boron trifluoride diethyl ether complex, boron trichloride, boron tribromide, ZnCl₂, SbF₅ or SbCl₅ to thereby isolate spiropentanes (hexanes) with general formula Ia: 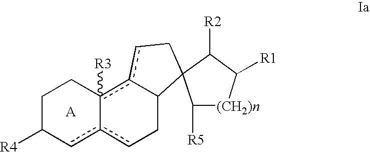
in which R₁=methyl or ethyl, R₂=OH, O, alkoxy (alkyl containing 1 to 12 C atoms), alkyloxymethyloxy (alkyl containing 1 to 3 C atoms), alkanoyloxy (alkyl containing 1 to 12 C atoms), aralkanoyloxy (alkyl containing 1 to 4 C atoms), aralkyloxy (alkyl containing 1 to 3 C atoms), aralkanoyloxy (alkyl containing 1 to 4 C atoms), R₃=methyl or H in the case in which A does not represent an aromatic ring and there is no double bond between C atoms 5 and 10 and 9 and 10; n=1 or 2; A=an aromatic, partially or completely hydrogenated ring and additional double bonds may be present between C atoms 4 and 5, 5 and 6, 5 and 10, 9 and 10 and/or 9 and 11; and
for the production of compounds of the invention of the 14β-fluoro, 15β-hydroxysteroid type, 17β-hydroxy-14β,15β-epoxysteroids with general formula II 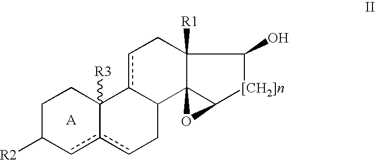
in which R₁=methyl or ethyl, R₂=O, OH, O-alkyl containing 1 to 12 C atoms, alkyloxymethyloxy (alkyl containing 1 to 3 C atoms), alkanoyloxy containing 1 to 12 C atoms, aralkanoyloxy (alkyl containing 1 to 4 C atoms) or aralkyloxy (alkyl containing 1 to 4 C atoms);
R₃=methyl or H in the case in which A does not designated an aromatic ring; n=1 or 2; A=an aromatic ring or a partially hydrogenated ring and additional double bonds may be present between C atoms 4 and 5, 5 and 6, and 9 and 11,
are converted, using a Lewis acid such as BF₃*Et₂O in an anhydrous aprotic solvent such as toluene, benzene, heptane, an ether or a halogenated hydrocarbon or corresponding mixtures of solvents and the resulting 14β-fluoro,15β-hydroxysteroids with general formula Ia: 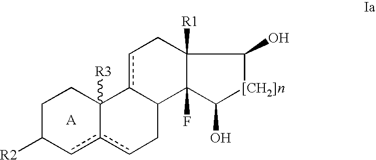
in which R₁, R₂ and R₃, n and A have the meanings given above and additional double bonds may be present between C atoms 4 and 5; 5 and 6 as well as 9 and 11, are isolated;
and/or may if necessary be derivatized or transformed in known manner;
wherein alcohols are oxidized, or are acylated, sulpamoylated, sulphated, phosphorylated, alkylated or silylated using an acid derivative, for example a chlorinated hydrocarbon chloroalkyl ester, or an alkylation agent in the presence of a base and a suitable solvent;
keto groups are protected by ketalization or thioketalization, and these and double bonds, and in the case of the Birch reduction the aromatic A ring as well, are reduced in known manner with complex hydrides or by catalytic hydrogenation;
double bonds are isomerized in known manner by treatment with bases or acids or are introduced by eliminating water from alcohols and enol ethers are cleaved by acids;
other double bonds in the vicinity of the aromatic A ring are introduced in a known manner by anodic oxidation, or additional double bonds are introduced by bromiding/dehydrobromiding;
aromatic ethers are cleaved in a known manner with boron tribromide or ethyl mercaptan and potassium ter-butylate or trimethylsilyl iodide;
the 17-keto function is converted in a known manner with Li/acetontrile or trimethylsulphonium iodide and subsequent treatment with a nucleophile to compounds containing a 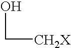
group (X=nucleophile);
wherein the order of reaction of successive derivatization steps and the number of reaction steps may be changed or adjusted and optionally, functional groups may be temporarily protected by ketalization, thioketalization or silylation.

The following examples serve to illustrate the invention without in any way limiting its scope.

### EXAMPLE 1

### 15α-hydroxy-3-methoxy-17-oxo-14β-methyl-13α-H-gona-1,3,5(10)-triene

0.5 ml of 17β-hydroxy-14α,15α-epoxy-3-methoxy-13β-methyl-gona-1,3,5(10)-triene was dissolved in about 3 ml of dry toluene and reacted with the exclusion of water at 65° C. with 0.55 mol of BF₃*Et₂O then stirred at this temperature for about 1 hour. After conversion, it was cooled to ambient temperature and supplemented with saturated bicarbonate solution. The product mixture was extracted with toluene to exhaustion, the organic extracts were washed with water to neutrality and then dried over sodium sulphate. After removing the solvent, a solid residue was obtained from which, following silica gel chromatography using a toluene-acetic ether mixture (5;1 to 3:1) as eluent following re-crystallization from chloroform/methanol, 30 g of 15α-hydroxy-3-methoxy-17-oxo-14β-methyl-13α-H-gona-1,3,5(10)-triene was isolated.

¹³C-NMR [ppm]: 126.1 (C1); 111.3 (C2); 157.2 (C3); 113.5 (C4); 137.6 (C5); 30.65 (C6); 23.05 (C7); 47.29 (C8); 38.68 (C9); 133.2 (C10); 29.32 (C11); 26.33 (C12); 45.46 (C13); 44.76 (C14); 76.27 (C15); 46.97 (C16); 220.4 (C17); 27.18 (14 CH₃); 54.99 (OCH₃);

IR [cm⁻¹]: 3610 (OH), 1728 (CO);

MS [m/z]: 323 [M+Na];

MP: 186° C. to 195° C. [α]_{D}: 67.55

### EXAMPLE 2

### 15α-hydroxy-3-methoxy-17-oxo-8α,14β-dimethyl-13α-H-gona-1,3,5(10),9(11)-tetraene

Step 1

### 17β-tert-butoxy-3-methoxy-13β-methyl-9(10)-seco-gona-1,3,5(10),8(14)-tetraene

44 mg (1.08 mmole) of 60% NaH-oil suspension and 200 mg (0.9 mmole) of dry Wiechert ketone was added to 10 ml of absolute glyme under argon. The mixture was stirred for 3 h at 65° C. and then supplemented with 335 mg (1.08 mmole) of 2(3-methoxyphenyl)-ethyl tosylate in 10 ml of absolute glyme and heated under reflux for 3 h at a temperature of 82° C. to 85° C. After cooling the solution, it was hydrolyzed with 15 ml of a saturated ammonium chloride solution and the seco product was extacted with diethyl ether to exhaustion. The combined ether extracts were washed with a bicarbonate solution and saturated salt solution and then dried over sodium sulphate. After removing the solvent under vacuum, the residue was chromatographed over silica gel. The eluent was toluene/acetic ether mixture between 50:1 and 10:1. 287 mg (0.74 mmole) (83% of theoretical yield) of 17β-tert-butoxy-3-methoxy-13β-methyl-9(10)-seco-gona-1,3,5(10),8(14)-tetraene was obtained.

¹³C-NMR [ppm]: 129 (C1); 110.9 (C2); 159.4 (C3); 114.8 (C4); 143.8 (C5); 34.6 (C6); 27.67 (C7); 131.6 (C8); 198.6 (C9); 121.4 (C10); 33.63 (C11); 34.15 (C12); 44.71 (C13); 169.7 (C14); 25.34 (C15); 29.76 (C16); 79.80 (C17); 15.76 (13 CH₃); 55.13 (OCH₃); 28.64 and 72.87 (t-Bu).

IR [cm⁻¹]: 1656 (CO);

MS [m/z]: 379 [M+Na];

[α]_{D}: +35.8° (MeOH).

Step 2

### 17β-tert-butoxy-3-methoxy-8α,13β-dimethyl-9(10)-seco-gona-1,3,5(10),14-tetraene

50 mg (0.14 mmole) of 17β-tert-butoxy-3-methoxy-13β-methyl-9(10)-seco-gona-1,3,5(10),8(14)-tetraene was dissolved in 3.5 ml of absolute glyme under argon and after adding 19 mg (0.168 mmole) of KtB, it was heated to 65° C. The red-brown solution was cooled to ambient temperature and immediately supplemented with 11 μl (0.17 mmole) of methyl iodide. After reaction, it was hydrolyzed with saturated ammonium chloride solution and extracted with diethyl ether to exhaustion. The ether extracts were combined, dried over sodium sulphate and evaporated off. The resulting crude product was chromatographed over silica gel using a toluene-acetic ether mixture (20:1). This produced 34 mg (0.092, 64% yield) of 17β-tert-butoxy-3-methoxy-8α,13β-dimethyl-9(10)-seco-gona-1,3,5(10),14-tetraene.

¹³C-NMR [ppm]: 129.4 (C1); 111.2 (C2); 159.7 (C3); 114.1 (C4); 143.7 (C5); 30.79 (C6); 40.62 (C7); 52.79 (C8); 214.5 (C9); 120.7 (C10); 34.99 (C11); 34.22 (C12); 46.3 (C13); 151.6 (C14); 122.4 (C15); 38.36 (C16); 81.08 (C17); 21.31 (8-CH₃); 17.58 (13-CH₃); 55.10 (OCH₃); 28.72 and 72.80 (t-Bu).

IR [cm⁻¹]: 1705 (CO);

MS [m/z]: 293 [M+Na];

[α]_{D}: +19.7° (MeOH).

Epoxidation, Variation B (Step 3)

### 17β-tert-butoxy-14α,15α-epoxy-3-methoxy-8α,13β-dimethyl-9(10)-seco-gona-1,3,5(10) triene (a)

### 17β-hydroxy-14α,15α-epoxy-3-methoxy-8α,13β-dimethyl-9(10)-seco-gona-1,3,5(10)-triene (b)

130 mg (0.675 mmole) of m-chloroperbenzoic acid was dissolved in 5 ml of methylene chloride at 0° C. and supplemented with 50 mg (0.135 mmole) of 17β-tert-butoxy-3-methoxy-8α,13β-dimethyl-9(10)-seco-gona-1,3,5(10),14-tetraene and stirred for 24 h at this temperature. After complete reaction of the educt, the reaction mixture was hydrolyzed with saturated sodium thiosulphate solution, with bicarbonate solution and lastly with water and then extracted to exhaustion with toluene. The combined organic extracts were washed to neutrality with bicarbonate solution and saturated NaCl solution, dried over sodium sulphate and evaporated. After chromatographing over silica gel using a toluene-acetic ether mixture (50:1 to 30:1), two products were isolated—29 mg (53% theoretical yield) of 17β-tert-butoxy-14α,15α-epoxy-3-methoxy-8α,13β-dimethyl-9(10)-seco-gona-1,3,5(10),14-triene (a) and 16 mg (35% theoretical yield) of 17β-hydroxy-14α,15α-epoxy-3-methoxy-8α,13β-dimethyl-9(10)-seco-gona-1,3,5(10)-triene (b).

(a) ¹³C-NMR [ppm]: 129.5 (C1); 111.4 (C2); 159.8 (C3); 114.1 (C4); 143.2 (C5); 31.44 (C6); 39.86 (C7); 52.17 (C8); 213.8 (C9); 120.6 (C10); 35.06 (C11); 30.92 (C12); 41.90 (C13); 73.17 (C14); 57.92 (C15); 35.46 (C16); 74.47 (C17); 17.89 (8-CH₃); 15.35 (13-CH₃); 55.17 (OCH₃); 28.56 and 72.19 (t-Bu).

IR [cm⁻¹]: 1703 (CO);

MS [m/z]: 409 [M+Na];

[α]_{D}: −1.250 (CHCl₃); MP: 68° C. to 73° C.

(b) [α]_{D}: +121.5° (CHCl₃); MP: 115° C. to 121° C.

Step 4

### 15α-hydroxy-3-methoxy-17-oxo-8α,14β-dimethyl-13α-H-gona-1,3,5(10),9(11)-tetraene

12 mg (0.031 mmole) of 17β-tert-butoxy-14α,15α-epoxy-3-methoxy-8α,13β-dimethyl-9(10)-seco-gona-1,3,5(10)-triene was dissolved in about 3 ml of dry toluene at 0° C., excluding water, and supplemented with 4.2 μl (0.034 mmole) of BF₃*Et₂O. After 2 min of reaction, the educt had been completely converted; it was hydrolyzed with saturated bicarbonate solution and the product was extracted with toluene to exhaustion. The combined organic extracts were washed to neutrality and dried over sodium sulphate. After removing the solvent, 8 mg of the product 14α-hydroxy-3-methoxy-17-oxo-8α,14β-dimethyl-13α-H-gona-1,3,5(10),9(11)-tetraene) was isolated in the form of an oil.

IR [cm⁻¹]: 1736 (17-keto);

MS [m/z]: 331 [M+H]; 353 [M+Na].

### EXAMPLE 2

### 3-methoxy-17-oxo-8α,14β-dimethyl-13α-H-gona-1,3,5(10),9(11),15-penta-ene

10 mg of 17β-hydroxy-14α,15α-epoxy-3-methoxy-8α,13β-dimethyl-9(10)-seco-gona-1,3,5(10)-triene (b) was converted in a manner analogous to that of Example 1 by converting the Wiechert ketone with 2(3-methoxyphenyl)-ethyl-tosylate or 2(3-methoxyphenyl)-ethyl bromide in accordance with steps 1 to 3 and then supplemented with BF₃*Et₂O in accordance with step 4. The reaction period was altered from 2 to 30 minutes and subsequently it was hydrolyzed with saturated bicarbonate solution and the reaction product was extracted to exhaustion with toluene. After the organic phase had been washed to neutrality, it was separated, dried over sodium sulphate and evaporated to dryness. 8 mg of the oily product was obtained.

IR [cm⁻¹]: 1715.

### EXAMPLE 3

### Spiro-[2,3,3a,4,5,9b-hexahydro-7-methoxy-1H-cyclopenta[a]naphthalene]-3,1′-[2′-S-methyl,3′-oxo-5′-R-hydroxyl-pentane]

30 mg (0.1 mmole) of 17β-hydroxy-14β,15β-epoxy-3-methoxy-13β-methyl-gona-1,3,5(10)-triene was suspended in 1 ml of toluene and pre-treated in an ultrasound bath to dissolve it better, with the exclusion of water. After adding 70 μl of BF₃*Et₂O at 0° C., the reaction mixture was slowly warmed to ambient temperature, with stirring. After successful transformation, a bicarbonate solution was added and the product mixture was extracted to exhaustion with toluene. The neutral organic solution was separated, dried over sodium sulphate and evaporated to dryness. After preparative thin layer chromatography (toluene/acetic ether 3:1) over silica (Merck), 24 mg of the spiro-cyclopentano compound was isolated, which could be crystallized from acetic ether and its structure could be determined by X ray analysis.

¹³C-NMR [ppm]: 127.6 (C1); 111.7 (C2); 157.9 (C3); 113.6 (C4); 137.5 (C5); 31.96 (C6); 25.5 (C7); 48.04 (C8); 44.14 (C9); 133.1 (C10); 29.18 (C11); 30.9 (C12); 45.44 (C13); 54.60 (C14); 74.85 (C15); 46.23 (C16); 218.4 (C17); 7.44 (C13).

IR [cm⁻¹]: 1726;

MS [m/z]: 323 [M+Na].

### EXAMPLE 4

### Spiro-[2,3,3a,4,5,9b-hexahydro-7-methoxy-1H-cyclopenta[a]naphthalene]-3,1′-[2′-S-methyl,3′-oxo-5′-R-hydroxyl-pentane]

30 mg (0.1 mmole) of 17α-hydroxy-14β,15β-epoxy-3-methoxy-13β-methyl-gona-1,3,5(10)-triene was suspended in 2 ml of toluene and pre-treated in an ultrasound bath to dissolve it better, with the exclusion of water. 100 μl of a saturated solution of anhydrous p-toluenesulphonic acid in toluene was then added and the reaction mixture was stirred at ambient temperature. After successfully transforming the educt, a bicarbonate solution was added and the product mixture was extracted to exhaustion with toluene. The organic solution, washed to neutrality, was separated, dried over sodium sulphate and evaporated to dryness. After silica chromatography (toluene/acetic ether 5:1 to 3:1), 15 mg of the spiro-cyclopentano compound was isolated, which could be crystallized from acetic ether.

IR [cm⁻¹]: 1726;

MS [m/z]: 323 [M+Na].

### EXAMPLE 6

### 15β,17β-dihydroxy-14β-fluoro-3-methoxy-13β-methyl-gona-1,3,5(10)-triene

50 mg of 17β-hydroxy-14β,15β-epoxy-3-methoxy-13β-methyl-gona-1,3,5(10)-triene was dissolved in 1 ml of toluene which had been dried over sodium, with the exclusion of water, and with heating, and after cooling to 0° C., supplemented with 70 μl of BF₃*Et₂O and stirred for 1 hr at 0° C. After successful transformation, it was hydrolyzed with bicarbonate solution and the product mixture was extracted with toluene. After chromatographic separation over silica using a toluene/acetic ether gradient of 5:1 to 3:1, after evaporation of the non-polar fraction, 15 mg of the fluoro product was isolated and crystallized from methylene chloride/methanol.

¹³C-NMR [ppm]: 126.9 (C1); 112.1 (C2); 157.8 (C3); 113.7 (C4); 137.8 (C5); 30.33 (C6); 22.56 (C7); 41 (C8)+; 39 (C9)+; 130.9 (C10); 26.27 (C11); 34 (C12)+; 48 (C13)+; 105/107 (C14)+; 72 (C15)+; 41.77 (C16); 79.44 (C17); 713.8 (13CH₃)+; 55.2 (OCH₃).

(signals marked “+” are split due to fluorine coupling)

IR [cm⁻¹]: 3595 (OH);

MS [m/z]: 320

MP: 188° C. to 191° C. [α]_{D}: +78.60

The invention will now be further described by the following numbered paragraphs:

1. 14β-alkyl-18-norsteroids with general formula I: 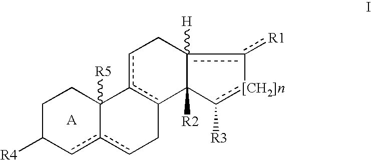
wherein
R₁=O, α- or β-OH, H, oxyalkyloxy (alkyl containing 2 to 5 C atoms), 17,2′-spirooxirane or is of the type 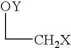
in which Y=H, alkyl (containing 1 to 6 C atoms), alkanoyl (containing 1 to 12 C atoms) and X=halogen, such as chorine, bromine, iodine or fluorine, pseudohalogen such as N₃, SCN, SeCN, CN, or alkyloxy (containing 1 to 6 C atoms) and alkanoyloxy (alkyl containing 1 to 12 C atoms), OSO₂NR₆R₇ with R₆ and R₇=H, alkyl (containing 1 to 6 C atoms) or cycloalkyl (containing 1 to 7 C atoms), or alkanoyl (alkyl containing 1 to 8 C atoms);
R₂=methyl or ethyl;
R₃=OH, H, OSO₂NR₆R₇ with R₆ and R₇=H, alkyl (containing 1 to 6 C atoms) or cycloalkyl (containing 1 to 7 C atoms), alkanoyl (alkyl containing 1 to 8 C atoms);
R₄=H, O, OH, O-alkyl containing 1 to 12 C atoms, alkyloxy-methyloxy (alkyl containing 1 to 3 C atoms), alkanoyloxy (containing 1 to 12 C atoms), aralkanoyloxy (alkyl containing 1 to 4 C atoms) or aralkyloxy (alkyl containing 1 to 4 C atoms), OSO₂NR₆R₇ with R₆ and R₇=H, alkyl (containing 1 to 6 C atoms), or cycloalkyl (containing 1 to 7 C atoms), alkanoyl (alkyl containing 1 to 8 C atoms), —OSO₂OR with R=H, alkyl or cycloalkyl (alkyl containing 1 to 7 C atoms); R₅=α or β H, methyl, chlorine or a methylene group bridging after C5 in the case in which A does not designate an aromatic ring or there is no double bond between C atoms 5 and 10;
n=1 or 2;
A=an aromatic ring or a partially or fully hydrogenated ring;
and additional double bonds may be present between C atoms 4 and 5; 5 and 6; 9 and 11; 8 and 9; 13 and 17 as well as 15 and 16;
as well as salts and esters with acids such as sulphuric acid, sulphamic acid, N-substituted sulphamic acid or phosphoric acids.

2. 8α,14β-dialkyl-18-norsteroids with general formula I: 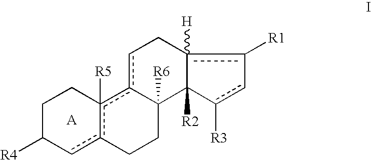
wherein
R₁=O, α- or β-OH, H, 17-spirooxirane or is of the type 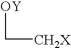
in which Y=H, alkyl (containing 1 to 6 C atoms), alkanoyl (containing 1 to 12 C atoms) and X=halogen, such as chorine, bromine, iodine or fluorine, pseudohalogen such as N₃, SCN, SeCN, CN, or alkyloxy (containing 1 to 6 C atoms) and alkanoyloxy (alkyl containing 1 to 12 C atoms), alkyloxy (alkyl containing 1 to 12 C atoms), alkyloxymethyloxy (alkyl containing 1 to 3 C atoms), alkanoyloxy (alkyl containing 1 to 12 C atoms), aralkyloxy (alkyl containing 1 to 4 C atoms), oxyalkyloxy (alkyl containing 2 to 6 C atoms), S—CH₂—CH₂S, O—CH₂—CH₂S, trialkylsilyloxy (alkyl containing 1 to 4 C atoms), OSO₂NR₆R₇ with R₆ and R₇=H, alkyl, cycloalkyl (containing 1 to 6 C atoms) or cycloalkyl (containing 1 to 7 C atoms), or alkanoyl (alkyl containing 1 to 8 C atoms);
R₂=methyl or ethyl;
R₃=H, O, OH, alkyloxy (alkyl containing 1 to 12 C atoms), alkyloxymethyloxy (alkyl containing 1 to 3 C atoms), alkanoyloxy (alkyl containing 1 to 12 C atoms), aralkanoyloxy (alkyl containing 1 to 4 C atoms) or aralkyloxy (alkyl containing 1 to 4 C atoms), OSO₂NR₆R₇ with R₆ and R₇=H, alkyl (containing 1 to 6 C atoms) or cycloalkyl (containing 1 to 7 C atoms) alkanoyl (alkyl containing 1 to 8 C atoms);
R₄=H, O, alkyloxy (alkyl containing 1 to 12 C atoms), alkyloxymethyloxy (alkyl containing 1 to 3 C atoms), aralkyloxy (alkyl containing 1 to 4 C atoms), alkanoyloxy (alkyl containing 1 to 12 C atoms), aralkanoyloxy (alkyl containing 1 to 4 C atoms) OSO₂NR₆R₇ with R₆ and R₇=H, alkyl (containing 1 to 6 C atoms) or cycloalkyl (containing 1 to 7 C atoms), alkanoyl (alkyl containing 1 to 8 C atoms);
R₅=α or β H, chlorine or a methylene group bridging after C5 in the case in which A does not designate an aromatic ring or there is no double bond between C atoms 5 and 10;
R₆=α-methyl, α-ethyl or α-methyoxymethyl;
A=an aromatic ring or a partially or fully hydrogenated ring;
and additional double bonds may be present between C atoms 4 and 5, 5 and 10, 9 and 10, 9 and 11, 13 and 17, 15 and 16, and/or 16 and 17;
as well as salts with amines and esters with acids such as sulphuric acid, sulphamic acid, N-substituted sulphamic acid or phosphoric acids.

3. Spiro[cyclopentano-perhydronaphthalene]-3,1′-pentanes (hexanes) with general formula I: 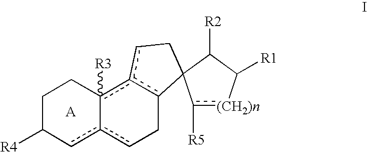
wherein
R₁=H, OH, O, O-alkyl (alkyl containing 1 to 12 C atoms), alkanoyloxy (alkyl containing 1 to 12 C atoms), alkyloxymethyloxy (alkyl containing 1 to 3 C atoms), aralkanoyloxy (alkyl containing 1 to 4 C atoms), oxyalkyloxy (alkyl containing 2 to 5 C atoms), S—CH₂—CH₂—S, O—CH₂—CH₂—S, 17-spiro-2′-oxirane, of the 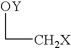
type with Y=H, alkyl (containing 1 to 6 C atoms), alkanoyl (alkyl containing 1 to 12 C atoms) and X=H, halogen such as chlorine, bromine, iodine or fluorine, pseudohalogen such as N₃, SCN, SeCN, CN or alkyloxy (containing 1 to 6 C atoms) and alkanoyloxy (alkyl containing 1 to 12 C atoms), OSO₂NR₆R₇ with R₆ and R₇=H, alkyl (containing 1 to 6 C atoms) or cycloalkyl (containing 1 to 7 C atoms) or alkanoyl (alkyl containing 1 to 8 C atoms);
R₂=methyl or ethyl and H;
R₃=methyl or H in the case in which A does not designate an aromatic ring and there is no double bond between C atoms 5 and 10 or 10 and 9;
R₄=H, OH, O, oxyalkyloxy (alkyl containing 2 to 5 C atoms), alkyloxy (alkyl containing 1 to 12 C atoms), alkyloxymethyloxy (alkyl containing 1 to 3 C atoms), alkanoyloxy (alkyl containing 1 to 12 C atoms), aralkyloxy (alkyl containing 1 to 3 C atoms), aralkanoyloxy (alkyl containing 1 to 4 C atoms), OSO₂NR₆R₇ with R₆ and R₇=H, alkyl (containing 1 to 6 C atoms) or cycloalkyl (containing 1 to 7 C atoms), alkanoyl (alkyl containing 1 to 8 C atoms);
R₅=H, OH, O, alkyloxy (alkyl containing 1 to 12 C atoms), alkoxymethyloxy (alkyl containing 1 to 3 C atoms), alkanoyloxy (alkyl containing 1 to 12 C atoms), aralkanoyloxy (alkyl containing 1 to 4 C atoms), OSO₂NR₆R₇ with R₆ and R₇=H, alkyl (containing 1 to 6 C atoms) or cycloalkyl (containing 1 to 7 C atoms), alkanoyl (alkyl containing 1 to 8 C atoms);
A=an aromatic ring or a partially or fully hydrogenated ring;
n=1 or 2;
and additional double bonds may be present between C atoms 4 and 5; 5 and 6, 8 and 9, 9 and 10, 5 and 10, 9 and 11, and/or 15 and 16;
as well as salts with amines and esters with acids such as sulphuric acid, sulphamic acid, N-substituted sulphamic acid or phosphoric acids.

4. 14β-fluoro-15β,17β-dihydroxy-steroids with general formula I: 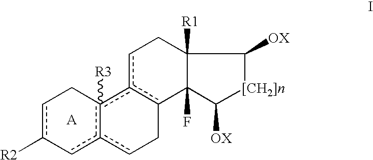
wherein
R₁=methyl or ethyl;
R₂=O, OH, O-alkyl (alkyl containing 1 to 12 C atoms), alkyloxymethyloxy (alkyl containing 1 to 3 C atoms), alkanoyloxy (alkyl containing 1 to 12 C atoms), aralkanoyloxy (alkyl containing 1 to 4 C atoms) or aralkyloxy (alkyl containing 1 to 4 C atoms), OSO₂NR₆R₇ with R₆ and R₇=H, alkyl (containing 1 to 6 C atoms) or cycloalkyl (containing 1 to 7 C atoms), alkanoyl (alkyl containing 1 to 8 C atoms), OSO₃H, OSO₃—, OSO₂R [in which R=alkyl or cycloakyl residue (alkyl containing 1 to 7 C atoms)];
R₃=methyl or H in the case in which A does not designate an aromatic ring or there is no double bond between C atoms 5 and 10 as well as 9 and 10;
X=H, SO₂NR₆R₇ [in which R₆ and R₇=H, alkyl (containing 1 to 6 C atoms) or cycloalkyl (containing 1 to 7 C atoms)], alkanoyl (alkyl containing 1 to 8 C atoms], alkyl (alkyl containing 1 to 12 C atoms), alkyloxymethyl (alkyl containing 1 to 3 C atoms), alkanoyl containing 1 to 12 C atoms, aralkanoyl (alkyl containing 1 to 4 C atoms) or aralkyl (alkyl containing 1 to 4 C atoms);
n=1 or 2;
A=an aromatic ring or a partially or fully hydrogenated ring;
and additional double bonds may be present between C atoms 2 and 3, 4 and 5; 5 and 6; 5 and 10, 9 and 10, 9 and 11; and 8 and 9;
as well as salts with amines and esters with acids such as sulphuric acid, sulphamic acid, N-substituted sulphamic acid or phosphoric acids.

5. 14β-alkyl-18-norsteroids according to paragraph 1, namely:
15α-hydroxy-3-methoxy-17-oxo-14β-methyl-13α-H-gona-1,3,5(10)-triene;
15α-hydroxy-3-methoxy-17-oxo-14β-methyl-13β-H-gona-1,3,5(10)-triene;
3,17β-dihydroxy-14β-methyl-13α-H-gona-1,3,5(10)-triene;
3,17β-disulphamoyloxy-14β-methyl-13α-H-gona-1,3,5(10)-triene;
3-hydroxy-14β-methyl-3-sulphamoyloxy-13α-H-gona-1,3,5(10)-triene;
17β-hydroxy-14β-methyl-3-sulphamoyloxy-13α-H-gona-1,3,5(10)-triene;
3-methoxy-14β-methyl-17-oxo-15α-sulphamoyloxy-13α-H-gona-1,3,5(10)-triene;
14β-methyl-17-oxo-3-sulphamoyloxy-13α-H-gona-1,3,5(10)-triene;
14β-methyl-3,17β,14α-trisulphamoyloxy-13α-H-gona-1,3,5(10)-triene;
N-[15α-methyleneoxycarbonyloxy-3-methoxy-15-methyl-17-oxo-13β-H-oestra-1,3,5(10)-triene]-N,N,N-triethyl ammonium iodide;
1 5α,17β-dihydroxy-14β-methyl-18-nor-13α-H-oestra-4-en-3-one;
1 5α,17β-disulphamoyloxy-14β-methyl-18-nor-13α-H-oestra-4-en-3-one.

6. 8α,14β-dialkyl-18-norsteroids according to paragraph 2, namely:
15α-hydroxy-3-methoxy-17-oxo-8α,14β-dimethyl-13α-H-gona-1,3,5(10),9(1)-tetraene;
3-methoxy-17-oxo-8α,14β-dimethyl-13α-H-gona-1,3,5(10),9(11),15-pentaene;
17β-hydroxy-3-methoxy-8α,14β-dimethyl-13α-H-gona-1,3,5(10)-triene;
17α-hydroxy-3-methoxy-8α,14β-dimethyl-13α-H-gona-1,3,5(10)-triene;
3,17β-dihydroxy-8α,14β-dimethyl-13α-H-gona-1,3,5(10)-triene;
3,17β-dihydroxy-8α,14β-dimethyl-13β-H-gona-1,3,5(10)-triene;
3,17β-dihydroxy-8α,14β-dimethyl-13α-H-gona-1,3,5(10),9(11)-tetraene;
3,17β-disulphamoyloxy-8α,14β-dimethyl-13β-H-gona-1,3,5(10)-triene;
N-[17α-methyleneoxycarbonyloxy-3-methoxy-8α,14β-dimethyl-13α-H-gona-1,3,5(10)-triene],
N,N,N-triethylammonium iodide.

7. Spiro(cyclopentano-perhydronaphthalene]-3,1′pentanes (hexanes) according to paragraph 3, namely:
spiro-[2,3,3a,4,5,9b-hexahydro-7-methoxy-1H-cyclopenta[a]naphthalene]-3,1′-[2′-S-methyl, 3′-oxo-5′-R-hydroxyl-pentane];
spiro-[2,3,3a,4,5,9b-hexahydro-7-methoxy-1H-cyclopenta[a]naphthalene]-3,1′-[2′-S-methyl, 3′-oxo-]-pentene];
spiro-[2,3,3a,4,5,9b-hexahydro-7-methoxy-1H-cyclopenta[a]naphthalene]-3,1′-[2′-S-methyl, 3′-oxo-5′-R-sulphamoyloxy]-pentane];
spiro-[2,3,3 α,4,5,9b-hexahydro-7-methoxy-1H-cyclopenta[a]naphthalene]-3,1′-[2′-S-methyl, 3′-oxo-]-pentene];
spiro-[2,3,3a,4,5,9b-hexahydro-7-sulphamoyloxy-1H-cyclopenta[a]naphthalene]-3,1′-[2′-S-methyl, 3′-oxo-]-pentene];
spiro-[2,3,3a,4,5,9b-hexahydro-7-sulphamoyloxy-1H-cyclopenta[a]naphthalene]-3,1′-[2′-S-ethyl, 3′-oxo-5′-R-sulphamoyloxy]-pentane];
N-{spiro-[2,3,3a,4,5,9b-hexahydro-7-methoxy-1H-cyclopenta[a]naphthalene]-3,1′-[2′-S-methyl,
3′-oxo-5′-methyleneoxycarbonyloxy]-pentane}, N,N,N-triethylammonium iodide.

8. 14β-fluoro-15β,17β-dihydroxy-steroids according to paragraph 4, namely:
14β-fluoro-15 β,17β-dihydroxy-3-methoxy-13β-methyl-gona-1,3,5(110)-triene;
14β-fluoro-3,15 β,17β-trihydroxy-13β-methyl-gona-1,3,5(10)-triene;
14β-fluoro-15β,17β-dihydroxy-19-nor-androsten-3-one;
14β-fluoro-3-methoxy-15β,17β-disulphamoyloxy-13β-methyl-gona-1,3,5(10)-triene;
14β-fluoro-15 β,17β-dihydroxy-3-sulphamoyloxy-13β-methyl-gona-1,3,5(10)-triene;
N-[17β-methyleneoxycarbonyloxy-3-methoxy-13β-methyl-gona-1,3,5(10)-triene]-N,N,N-triethylammonium iodide.

9. A method for the preparation of 14β-methyl-18-norsteroids with general formula I in accordance with paragraphs 1 and 5, characterized in that 17β-hydroxy-14α,15α-epoxysteroids with general formula II: 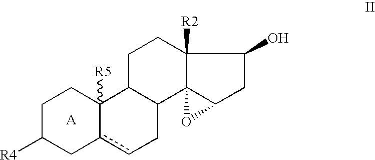
in which R₂=methyl or ethyl;
R₄=H, O, OH, O-alkyl containing 1 to 12 C atoms, alkyloxymethyloxy (alkyl containing 1 to 3 C atoms), alkanoyloxy containing 1 to 12 C atoms, aralkanoyloxy (alkyl containing 1 to 4 C atoms) or aralkyloxy (alkyl containing 1 to 4 C atoms);
R₅=methyl or H in the case in which A does not designate an aromatic ring;
A=an aromatic ring or a partially hydrogenated ring; and
may contain additional double bonds between C atoms 4 and 5; 5 and 6;
are converted in an aprotic solvent such as toluene in the presence of an anhydrous acid such as p-toluenesulphonic acid or a Lewis acid such as boron trifluoride diethyl ether complex, with heating, to thereby isolate 14β-alkyl-18-norsteroids with general formula I: 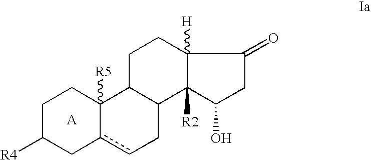
in which
R₂=methyl or ethyl;
R₄=H, O, OH, O-alkyl containing 1 to 12 C atoms, alkyloxymethyloxy (alkyl containing 1 to 3 C atoms), alkanoyloxy containing 1 to 12 C atoms, aralkanoyloxy (alkyl containing 1 to 4 C atoms) or aralkyloxy (alkyl containing 1 to 4 C atoms);
R₅=methyl or H in the case in which A does not designate an aromatic ring;
A=an aromatic ring or a partially hydrogenated ring;
and additional double bonds may be present between C atoms 4 and 5; 5 and 6, and/or if appropriate to derivatize them in a manner which is known per se;
whereby alcohols are oxidized, or are acylated, sulpamoylated, sulphated, phosphorylated, alkylated or silylated using an acid derivative, an alkylation agent, or a silylating agent in the presence of a base and a suitable solvent;
keto groups, and in the case of the Birch reduction the aromatic A ring as well, are reduced in known manner or are protected by ketalization or thioketalization;
double bonds are displaced by treatment with bases or acids, enol ethers are cleaved by acids and double bonds are introduced by eliminating water from alcohols;
other double bonds in the vicinity of the aromatic A ring are introduced in a known manner by anodic oxidation, or additional double bonds are introduced by bromiding/dehydrobromiding;
aromatic ethers are cleaved in a known manner with boron tribromide or ethyl mercaptan and potassium ter-butylate;
the 17-keto function is converted in a known manner with Li/acetontrile or trimethylsulphonium iodide and subsequent treatment with a nucleophile to form compounds containing a 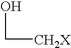
(X=nucleophile);
wherein the order of reaction steps in the derivatization and the number of reaction steps may be changed or adjusted.

10. A method for the production of 8α,14β-dialkyl-18-norsteroids with general formula I according to paragraph 2 and paragraph 6, characterized in that the Weichert ketone (II) is pre-treated in an ether such as dimethoxyethane or tetrahydrofuran with a base such as sodium hydride or potassium tert-butylate and allowed to react with an alkylation synthone (III) such as 2(3-alkyloxyphenyl)-tosylate or -bromide and is transformed, after further action with a base using an alkylation agent such as methyl, ethyl or methoxymethyl iodide, into 17β-tert-butoxy-8α-alkyl-9-oxo-9,10-seco-gona-1,3,5(10),14-tetraene (IV): 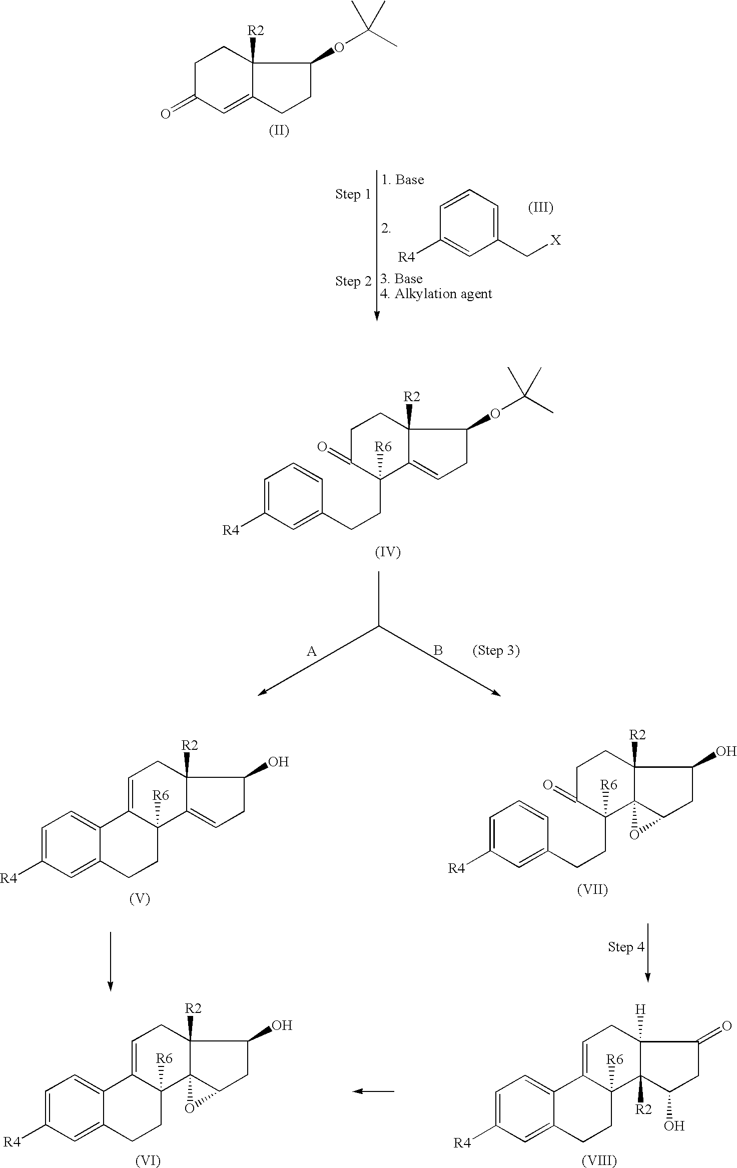
in which
R₂=methyl or ethyl;
R₄=alkyloxy (alkyl containing 1 to 12 C atoms), alkyloxymethyloxy (alkyl containing 1 to 3 C atoms);
R₆=methyl, ethyl or alkyloxymethyloxy (alkyl containing 1 to 3 C atoms);
X=tosyloxy, bromide, chloride or iodide;
and this is isolated either via route A:
transformation by aldol condensation using an organic acid such as p-toluenesulphonic acid or a Lewis acid such as boron trifluoride diethyl ether complex in an aprotic solvent into 17β-hydroxy-8α-alkyl-gona-1,3,5(10),9(11),14-pentaene with general formula (V), in which R₄ and R₂ have the meanings defined above, the latter being reduced, by selective epoxidation using peracids such as m-chloroperbenzoic acid or by Sharpless epoxidation using vanadylacetyl acetonate and tert-butyl hydroperoxide in any solvent such as toluene, to the 14α,15α-epoxide with general formula (VI), this latter being transformed, by treatment with a Lewis acid such as boron trifluoride diethyl ether complex in a suitable anhydrous solvent such as toluene, into 8α,14β-dialkyl-15α-hydroxy-17-oxo-gona-1,3,5(10),9(11)-tetraene (I or VIII with the meanings defined above for R₂ and R₄);
or via route B:
reduction to the seco-14α,15α-epoxide (VII) by selective epoxidation using peracids, which, by means of an organic acid or Lewis acid such as boron trifluoride diethyl ether complex in a suitable anhydrous solvent such as toluene, is transformed into 8α,14β-dialkyl-15α-hydroxy-17-oxo-gona-1,3,5(10),9(11)-tetraene (I or VIII with the meanings defined above for R₂ and R₄);
and/or this may if appropriate be transformed or derivatized in a manner which is known per se;
whereby alcohols are oxidized, or are acylated, sulpamoylated, sulphated, phosphorylated, alkylated or silylated using an acid derivative, for example chlorinated hydrocarbon chloroalkyl ester, an alkylation agent, or a silylating agent in the presence of a base and a suitable solvent;
keto groups are protected by ketalization or thioketalization, and these as well as double bonds, in the case of the Birch reduction the aromatic A ring as well, are reduced in known manner with complex hydrides or by catalytic hydrogenation;
double bonds are isomerized in known manner by treatment with bases or acids or are introduced by eliminating water from alcohols and enol ethers are cleaved by acids;
other double bonds in the vicinity of the aromatic A ring are introduced in a known manner by anodic oxidation, or additional double bonds are introduced by bromiding/dehydrobromiding;
aromatic and aliphatic ethers are cleaved in a known manner with boron tribromide or ethyl mercaptan and potassium ter-butylate or trimethylsilyl iodide;
the 17-keto function is converted in a known manner with Li/acetontrile or trimethylsulphonium iodide and subsequent treatment with a nucleophile to form compounds containing a 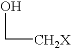
group
(X=nucleophile);
wherein the order of reaction successive steps in the derivatization steps and the number of reaction steps may be changed or adjusted.

11. A method for the production of spiro[cyclopentanoperhydronaphthalene]-3,1′-pentanes (-hexanes) with general formula I according to paragraph 3 and paragraph 7, characterized in that 17α-hydroxy-14β,15β-epoxysteroids with general formula II: 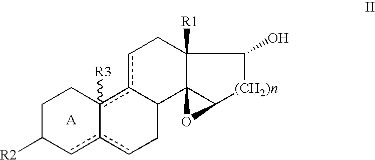
in which
R₁=methyl or ethyl;
R₂=OH, O, alkoxy (alkyl containing 1 to 12 C atoms), alkyloxymethyloxy (alkyl containing 1 to 3 C atoms), alkanoyloxy (alkyl containing 1 to 12 C atoms), aralkanoyloxy (alkyl containing 1 to 4 C atoms), aralkyloxy (alkyl containing 1 to 3 C atoms), aralkanoyloxy (alkyl containing 1 to 4 C atoms);
R₃=methyl or H in the case in which A does not represent an aromatic ring and there is no double bond between C atoms 5 and 10 and 9 and 10;
n=1 or 2;
A=an aromatic, partially or completely hydrogenated ring;
and additional double bonds may be present between C atoms 4 and 5, 5 and 6, 5 and 10, 9 and 10 and/or 9 and 11;
in an anhydrous aprotic solvent such as toluene, are converted in the presence of an acid such as p-toluenesulphonic acid or a Lewis acid such as boron trifluoride, to isolate spiropentanes (hexanes) with general formula Ia: 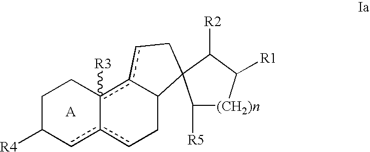
in which
R₁=methyl or ethyl;
R₂=OH, O, alkoxy (alkyl containing 1 to 12 C atoms), alkyloxymethyloxy (alkyl containing 1 to 3 C atoms), alkanoyloxy (alkyl containing 1 to 12 C atoms), aralkanoyloxy (alkyl containing 1 to 4 C atoms), aralkyloxy (alkyl containing 1 to 3 C atoms), aralkanoyloxy (alkyl containing 1 to 4 C atoms);
R₃=methyl or H in the case in which A does not represent an aromatic ring and there is no double bond between C atoms 5 and 10 and 9 and 10;
n=1 or 2;
A=an aromatic or partially hydrogenated ring;
and additional double bonds may be present between C atoms: 4 and 5, 5 and 6, 5 and 10, 9 and 10 and/or 9 and 11;
and/or they may, if appropriate, be transformed in a manner which is known per se;
whereby alcohols are oxidized, or are acylated, sulpamoylated, sulphated, phosphorylated, alkylated or silylated using an acid derivative, an alkylation agent, or a silylating agent in the presence of a base and a suitable solvent;
keto groups, and in the case of the Birch reduction the aromatic A ring as well, are reduced in known manner with complex hydrides or by catalytic hydrogenation or are protected by ketalization or thioketalization;
double bonds are isomerized by treatment with bases or acids, enol ethers are cleaved by acids and double bonds are introduced by eliminating water from alcohols;
other double bonds in the vicinity of the aromatic A ring are introduced in a known manner by anodic oxidation, or additional double bonds are introduced by bromiding/dehydrobromiding;
aromatic and aliphatic ethers are cleaved in a known manner with boron tribromide or ethyl mercaptan and potassium ter-butylate or trimethylsilyl iodide;
the 17-keto function is converted in a known manner with Li/acetontrile or trimethylsulphonium iodide and subsequent treatment with a nucleophile to form compounds containing a 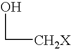
(X=nucleophile);
wherein the order of successive reaction steps in the derivatization steps and the number of reaction steps may be changed or adjusted.

12. A method for the production of 14β-fluoro, 15β,17β-dihydroxysteroids with general formula I according to paragraph 4 and paragraph 8, characterized in that 17β-hydroxy-14β,15β-epoxysteroids with general formula II: 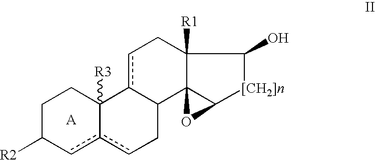
in which
R₁=methyl or ethyl;
R₂=O, OH, O-alkyl containing 1 to 12 C atoms, alkyloxymethyloxy (alkyl containing 1 to 3 C atoms), alkanoyloxy containing 1 to 12 C atoms, aralkanoyloxy (alkyl containing 1 to 4 C atoms) or aralkyloxy (alkyl containing 1 to 4 C atoms);
R₃=methyl or H in the case in which A does not represent an aromatic ring;
n=1 or 2;
A=an aromatic ring or a partially hydrogenated ring;
and additional double bonds may be present between C atoms 4 and 5; 5 and 6 and 9 and 11;
is converted by means of BF₃*Et₂O in an anhydrous aprotic solvent such as toluene and the resulting 14β-fluoro,15β-hydroxysteroids with general formula Ia: 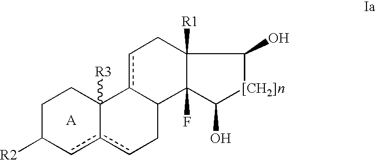
in which
R₁=methyl or ethyl;
R₂=O, OH, O-alkyl (alkyl containing 1 to 12 C atoms), alkyloxymethyloxy (alkyl containing 1 to 3 C atoms), alkanoyloxy (alkyl containing 1 to 12 C atoms), aralkanoyloxy (alkyl containing 1 to 4 C atoms) or aralkyloxy (alkyl containing 1 to 4 C atoms);
R₃=methyl or H in the case in which A does not represent an aromatic ring;
n=1 or 2;
A=an aromatic ring or a partially hydrogenated ring;
and additional double bonds may be present between C atoms: 4 and 5; 5 and 6 and 9 and 11, is isolated;
and/or it is derivatized or transformed in known manner;
whereby alcohols are acylated, sulpamylated, sulphated, phosphorylated or alkylated using an acid derivative or an alkylation agent in the presence of a proton-abstracting base and a suitable solvent;
the aromatic A ring is reduced in known manner using the Birch reduction and converted into 3-keto-Δ⁴- or 3-keto-5(10) compounds by acid treatment;
double bonds in the vicinity of the aromatic A ring are introduced in a known manner by anodic oxidation, or additional double bonds are introduced by bromiding/dehydrobromiding from 3-keto-5(10 compounds;
keto groups are reduced in known manner using complex hydrides or by catalytic hydrogenation;
aromatic and aliphatic ethers are cleaved in a known manner with boron tribromide, ethyl mercaptan and potassium ter-butylate or trimethylsilyl iodide;
wherein the order of successive reaction steps in the derivatization steps and the number of reaction steps may be changed or adjusted.

13. Pharmaceutical compositions containing at least one 14β-alkyl-18-norsteroid according to paragraph 1 and paragraph 5, a 8α,14β-dialkyl-18-norsteroid in accordance with paragraph 2 and paragraph 6, a spiro[cyclopentano-perhydronaphthalene]-3,1′-pentane (-hexane) according to paragraph 3 and paragraph 7 or a 14β-fluoro-15β,17β-dihydroxysteroid according to paragraph 4 and paragraph 8, optionally with pharmaceutically acceptable excipients, carriers and/or stabilizers.

14. Pharmaceutical preparations according to one of the preceding paragraphs, characterized in that administration is by systemic or topical, oral, subcutaneous or dermal means or by injection.

1. 14β-alkyl-18-norsteroids with general formula I: 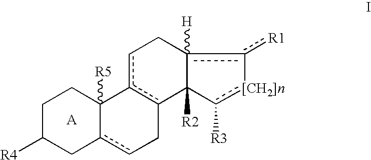
wherein
R₁=O, α- or β-OH, H, oxyalkyloxy (alkyl containing 2 to 5 C atoms), 17,2′-spirooxirane or is of the type 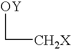
in which Y=H, alkyl (containing 1 to 6 C atoms), alkanoyl (containing 1 to 12 C atoms) and X=halogen, such as chorine, bromine, iodine or fluorine, pseudohalogen such as N₃, SCN, SeCN, CN, or alkyloxy (containing 1 to 6 C atoms) and alkanoyloxy (alkyl containing 1 to 12 C atoms), OSO₂NR₆R₇ with R₆ and R₇=H, alkyl (containing 1 to 6 C atoms) or cycloalkyl (containing 1 to 7 C atoms), or alkanoyl (alkyl containing 1 to 8 C atoms);
R₂=methyl or ethyl;
R₃=OH, H, OSO₂NR₆R₇ with R₆ and R₇=H, alkyl (containing 1 to 6 C atoms) or cycloalkyl (containing 1 to 7 C atoms), alkanoyl (alkyl containing 1 to 8 C atoms);
R₄=H, O, OH, O-alkyl containing 1 to 12 C atoms, alkyloxy-methyloxy (alkyl containing 1 to 3 C atoms), alkanoyloxy (containing 1 to 12 C atoms), aralkanoyloxy (alkyl containing 1 to 4 C atoms) or aralkyloxy (alkyl containing 1 to 4 C atoms), OSO₂NR₆R₇ with R₆ and R₇=H, alkyl (containing 1 to 6 C atoms), or cycloalkyl (containing 1 to 7 C atoms), alkanoyl (alkyl containing 1 to 8 C atoms), —OSO₂OR with R=H, alkyl or cycloalkyl (alkyl containing 1 to 7 C atoms);
R₅=α or β H, methyl, chlorine or a methylene group bridging after C5 in the case in which A does not designate an aromatic ring or there is no double bond between C atoms 5 and 10;
n=1 or 2;
A=an aromatic ring or a partially or fully hydrogenated ring;
and additional double bonds may be present between C atoms 4 and 5; 5 and 6; 9 and 11; 8 and 9; 13 and 17 as well as 15 and 16;
as well as salts and esters with acids such as sulphuric acid, sulphamic acid, N-substituted sulphamic acid or phosphoric acids.

2. 8α,14β-dialkyl-18-norsteroids with general formula I: 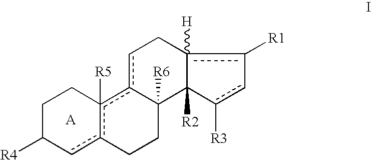
wherein
R₁=O, α- or β-OH, H, 17-spirooxirane or is of the type 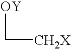
in which Y=H, alkyl (containing 1 to 6 C atoms), alkanoyl (containing 1 to 12 C atoms) and X=halogen, such as chorine, bromine, iodine or fluorine, pseudohalogen such as N₃, SCN, SeCN, CN, or alkyloxy (containing 1 to 6 C atoms) and alkanoyloxy (alkyl containing 1 to 12 C atoms), alkyloxy (alkyl containing 1 to 12 C atoms), alkyloxymethyloxy (alkyl containing 1 to 3 C atoms), alkanoyloxy (alkyl containing 1 to 12 C atoms), aralkyloxy (alkyl containing 1 to 4 C atoms), oxyalkyloxy (alkyl containing 2 to 6 C atoms), S—CH₂—CH₂S, O—CH₂—CH₂S, trialkylsilyloxy (alkyl containing 1 to 4 C atoms), OSO₂NR₆R₇ with R₆ and R₁=H, alkyl, cycloalkyl (containing 1 to 6 C atoms) or cycloalkyl (containing 1 to 7 C atoms), or alkanoyl (alkyl containing 1 to 8 C atoms);
R₂=methyl or ethyl;
R₃=H, O, OH, alkyloxy (alkyl containing 1 to 12 C atoms), alkyloxymethyloxy (alkyl containing 1 to 3 C atoms), alkanoyloxy (alkyl containing 1 to 12 C atoms), aralkanoyloxy (alkyl containing 1 to 4 C atoms) or aralkyloxy (alkyl containing 1 to 4 C atoms), OSO₂NR₆R₇ with R₆ and R₇=H, alkyl (containing 1 to 6 C atoms) or cycloalkyl (containing 1 to 7 C atoms) alkanoyl (alkyl containing 1 to 8 C atoms);
R₄=H, O, alkyloxy (alkyl containing 1 to 12 C atoms), alkyloxymethyloxy (alkyl containing 1 to 3 C atoms), aralkyloxy (alkyl containing 1 to 4 C atoms), alkanoyloxy (alkyl containing 1 to 12 C atoms), aralkanoyloxy (alkyl containing 1 to 4 C atoms) OSO₂NR₆R₇ with R₆ and R₇=H, alkyl (containing 1 to 6 C atoms) or cycloalkyl (containing 1 to 7 C atoms), alkanoyl (alkyl containing 1 to 8 C atoms);
R₅=α or β H, chlorine or a methylene group bridging after C5 in the case in which A does not designate an aromatic ring or there is no double bond between C atoms 5 and 10;
R₆=α-methyl, α-ethyl or α-methyoxymethyl;
A=an aromatic ring or a partially or fully hydrogenated ring;
and additional double bonds may be present between C atoms 4 and 5, 5 and 10, 9 and 10, 9 and 11, 13 and 17, 15 and 16, and/or 16 and 17;
as well as salts with amines and esters with acids such as sulphuric acid, sulphamic acid, N-substituted sulphamic acid or phosphoric acids.

3. Spiro[cyclopentano-perhydronaphthalene]-3,1′-pentanes (hexanes) with general formula I: 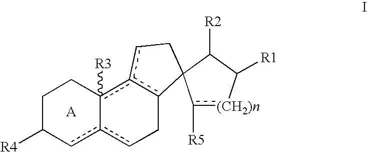
wherein
R₁=H, OH, O, O-alkyl (alkyl containing 1 to 12 C atoms), alkanoyloxy (alkyl containing 1 to 12 C atoms), alkyloxymethyloxy (alkyl containing 1 to 3 C atoms), aralkanoyloxy (alkyl containing 1 to 4 C atoms), oxyalkyloxy (alkyl containing 2 to 5 C atoms), S—CH₂—CH₂—S, O—CH₂—CH₂—S, 17-spiro-2′-oxirane, of the 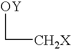
type with Y=H, alkyl (containing 1 to 6 C atoms), alkanoyl (alkyl containing 1 to 12 C atoms) and X=H, halogen such as chlorine, bromine, iodine or fluorine, pseudohalogen such as N₃, SCN, SeCN, CN or alkyloxy (containing 1 to 6 C atoms) and alkanoyloxy (alkyl containing 1 to 12 C atoms), OSO₂NR₆R₇ with R₆ and R₇=H, alkyl (containing 1 to 6 C atoms) or cycloalkyl (containing 1 to 7 C atoms) or alkanoyl (alkyl containing 1 to 8 C atoms);
R₂=methyl or ethyl and H;
R₃=methyl or H in the case in which A does not designate an aromatic ring and there is no double bond between C atoms 5 and 10 or 10 and 9;
R₄=H, OH, O, oxyalkyloxy (alkyl containing 2 to 5 C atoms), alkyloxy (alkyl containing 1 to 12 C atoms), alkyloxymethyloxy (alkyl containing 1 to 3 C atoms), alkanoyloxy (alkyl containing 1 to 12 C atoms), aralkyloxy (alkyl containing 1 to 3 C atoms), aralkanoyloxy (alkyl containing 1 to 4 C atoms), OSO₂NR₆R₇ with R₆ and R₇=H, alkyl (containing 1 to 6 C atoms) or cycloalkyl (containing 1 to 7 C atoms), alkanoyl (alkyl containing 1 to 8 C atoms);
R₅=H, OH, O, alkyloxy (alkyl containing 1 to 12 C atoms), alkoxymethyloxy (alkyl containing 1 to 3 C atoms), alkanoyloxy (alkyl containing 1 to 12 C atoms), aralkanoyloxy (alkyl containing 1 to 4 C atoms), OSO₂NR₆R₇ with R₆ and R₇=H, alkyl (containing 1 to 6 C atoms) or cycloalkyl (containing 1 to 7 C atoms), alkanoyl (alkyl containing 1 to 8 C atoms);
A=an aromatic ring or a partially or fully hydrogenated ring;
n=1 or 2;
and additional double bonds may be present between C atoms 4 and 5; 5 and 6, 8 and 9, 9 and 10, 5 and 10, 9 and 11, and/or 15 and 16;
as well as salts with amines and esters with acids such as sulphuric acid, sulphamic acid, N-substituted sulphamic acid or phosphoric acids.

4. 14β-fluoro-15β,17β-dihydroxy-steroids with general formula I: 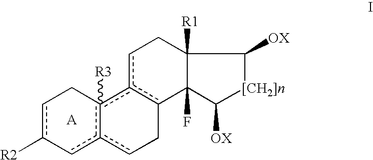
wherein
R₁=methyl or ethyl;
R₂=O, OH, O-alkyl (alkyl containing 1 to 12 C atoms), alkyloxymethyloxy (alkyl containing 1 to 3 C atoms), alkanoyloxy (alkyl containing 1 to 12 C atoms), aralkanoyloxy (alkyl containing 1 to 4 C atoms) or aralkyloxy (alkyl containing 1 to 4 C atoms), OSO₂NR₆R₇ with R₆ and R₇=H, alkyl (containing 1 to 6 C atoms) or cycloalkyl (containing 1 to 7 C atoms), alkanoyl (alkyl containing 1 to 8 C atoms), OSO₃H, OSO₃—, OSO₂R [in which R=alkyl or cycloakyl residue (alkyl containing 1 to 7 C atoms)];
R₃=methyl or H in the case in which A does not designate an aromatic ring or there is no double bond between C atoms 5 and 10 as well as 9 and 10;
X=H, SO₂NR₆R₇ [in which R₆ and R₇=H, alkyl (containing 1 to 6 C atoms) or cycloalkyl (containing 1 to 7 C atoms)], alkanoyl (alkyl containing 1 to 8 C atoms], alkyl (alkyl containing 1 to 12 C atoms), alkyloxymethyl (alkyl containing 1 to 3 C atoms), alkanoyl containing 1 to 12 C atoms, aralkanoyl (alkyl containing 1 to 4 C atoms) or aralkyl (alkyl containing 1 to 4 C atoms);
n=1 or 2;
A=an aromatic ring or a partially or fully hydrogenated ring;
and additional double bonds may be present between C atoms 2 and 3, 4 and 5; 5 and 6; 5 and 10, 9 and 10, 9 and 11; and 8 and 9;
as well as salts with amines and esters with acids such as sulphuric acid, sulphamic acid, N-substituted sulphamic acid or phosphoric acids.

5. 14β-alkyl-18-norsteroids according to claim 1, namely:
15α-hydroxy-3-methoxy-17-oxo-14β-methyl-13α-H-gona-1,3,5(10)-triene;
15α-hydroxy-3-methoxy-17-oxo-14β-methyl-13β-H-gona-1,3,5(110)-triene;
3,17β-dihydroxy-14′-methyl-13α-H-gona-1,3,5(10)-triene;
3,17β-disulphamoyloxy-14β-methyl-13α-H-gona-1,3,5(10)-triene;
3-hydroxy-14β-methyl-3-sulphamoyloxy-13α-H-gona-1,3,5(10)-triene;
17β-hydroxy-14β-methyl-3-sulphamoyloxy-13α-H-gona-1,3,5(10)-triene;
3-methoxy-14β-methyl-17-oxo-15α-sulphamoyloxy-13α-H-gona-1,3,5(10)-triene;
14β-methyl-17-oxo-3-sulphamoyloxy-13α-H-gona-1,3,5(10)-triene;
14β-methyl-3,17β,14α-trisulphamoyloxy-13α-H-gona-1,3,5(10)-triene;
N-[15α-methyleneoxycarbonyloxy-3-methoxy-15β-methyl-17-oxo-13β-H-oestra-1,3,5(10)-triene]-N,N,N-triethyl ammonium iodide;
15α,17′-dihydroxy-14β-methyl-18-nor-13α-H-oestra-4-en-3-one;
15α,17β-disulphamoyloxy-14′-methyl-18-nor-13α-H-oestra-4-en-3-one.

6. 8α,14β-dialkyl-18-norsteroids according to claim 2, namely:
15α-hydroxy-3-methoxy-17-oxo-8α,14β-dimethyl-13α-H-gona-1,3,5(10),9(11)-tetraene;
3-methoxy-17-oxo-8α,14β-dimethyl-13α-H-gona-1,3,5(10),9(11),15-pentaene;
17β-hydroxy-3-methoxy-8α,14β-dimethyl-13α-H-gona-1,3,5(10)-triene;
17α-hydroxy-3-methoxy-8α,14β-dimethyl-13α-H-gona-1,3,5(10)-triene;
3,17β-dihydroxy-8α,14β-dimethyl-13α-H-gona-1,3,5(10)-triene;
3,17β-dihydroxy-8α,14β-dimethyl-13β-H-gona-1,3,5(10)-triene;
3,17β-dihydroxy-8α,14β-dimethyl-13α-H-gona-1,3,5(10),9(11)-tetraene;
3,17β-disulphamoyloxy-8α,14β-dimethyl-13β-H-gona-1,3,5(10)-triene;
N-[17α-methyleneoxycarbonyloxy-3-methoxy-8α,14β-dimethyl-13α-H-gona-1,3,5(10)-triene],
N,N,N-triethylammonium iodide.

7. Spiro(cyclopentano-perhydronaphthalene]-3,1′pentanes (hexanes) according to claim 3, namely:
spiro-[2,3,3a,4,5,9b-hexahydro-7-methoxy-1H-cyclopenta[a]naphthalene]-3,1′-[2′-S-methyl, 3′-oxo-5′-R-hydroxyl-pentane];
spiro-[2,3,3a,4,5,9b-hexahydro-7-methoxy-1H-cyclopenta[a]naphthalene]-3,1′-[2′-S-methyl, 3′-oxo-]-pentene];
spiro-[2,3,3a,4,5,9b-hexahydro-7-methoxy-1H-cyclopenta[a]naphthalene]-3,1′-[2′-S-methyl, 3′-oxo-5′-R-sulphamoyloxy]-pentane];
spiro-[2,3,3a,4,5,9b-hexahydro-7-methoxy-1H-cyclopenta[a]naphthalene]-3,1′-[2′-S-methyl, 3′-oxo-]-pentene];
spiro-[2,3,3a,4,5,9b-hexahydro-7-sulphamoyloxy-1H-cyclopenta[a]naphthalene]-3,1′-[2′-S-methyl, 3′-oxo-]-pentene];
spiro-[2,3,3a,4,5,9b-hexahydro-7-sulphamoyloxy-1H-cyclopenta[a]naphthalene]-3,1′-[2′-S-methyl, 3′-oxo-5′-R-sulphamoyloxy]-pentane];
N-{spiro-[2,3,3a,4,5,9b-hexahydro-7-methoxy-1H-cyclopenta[a]naphthalene]-3,1′-[2′-S-methyl,
3′-oxo-5′-methyleneoxycarbonyloxy]-pentane}, N,N,N-triethylammonium iodide.

8. 14β-fluoro-15 β,17β-dihydroxy-steroids according to claim 4, namely:
14β-fluoro-15β,17-dihydroxy-3-methoxy-13β-methyl-gona-1,3,5(10)-triene;
14β-fluoro-3,15β,17β-trihydroxy-13β-methyl-gona-1,3,5(10)-triene;
14β-fluoro-15,17β-dihydroxy-19-nor-androsten-3-one;
14β-fluoro-3-methoxy-15β,17β-disulphamoyloxy-13β-methyl-gona-1,3,5(10)-triene;
14β-fluoro-15β,17β-dihydroxy-3-sulphamoyloxy-13β-methyl-gona-1,3,5(10)-triene;
N-[17β-methyleneoxycarbonyloxy-3-methoxy-13β-methyl-gona-1,3,5(10)-triene]-N,N,N-triethylammonium iodide.

9. A method for the preparation of 14β-methyl-18-norsteroids with general formula I in accordance with claim 1, characterized in that 17β-hydroxy-14α,15α-epoxysteroids with general formula II: 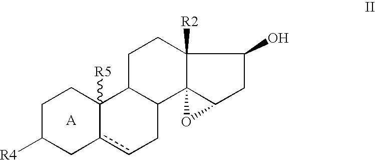
in which R₂=methyl or ethyl;
R₄=H, O, OH, O-alkyl containing 1 to 12 C atoms, alkyloxymethyloxy (alkyl containing 1 to 3 C atoms), alkanoyloxy containing 1 to 12 C atoms, aralkanoyloxy (alkyl containing 1 to 4 C atoms) or aralkyloxy (alkyl containing 1 to 4 C atoms);
R₅=methyl or H in the case in which A does not designate an aromatic ring;
A=an aromatic ring or a partially hydrogenated ring; and
may contain additional double bonds between C atoms 4 and 5; 5 and 6;
are converted in an aprotic solvent such as toluene in the presence of an anhydrous acid such as p-toluenesulphonic acid or a Lewis acid such as boron trifluoride diethyl ether complex, with heating, to thereby isolate 14β-alkyl-18-norsteroids with general formula I: 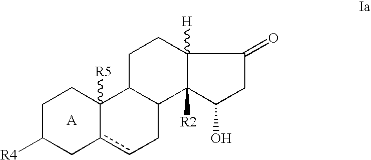
in which
R₂=methyl or ethyl;
R₄=H, O, OH, O-alkyl containing 1 to 12 C atoms, alkyloxymethyloxy (alkyl containing 1 to 3 C atoms), alkanoyloxy containing 1 to 12 C atoms, aralkanoyloxy (alkyl containing 1 to 4 C atoms) or aralkyloxy (alkyl containing 1 to 4 C atoms);
R₅=methyl or H in the case in which A does not designate an aromatic ring;
A=an aromatic ring or a partially hydrogenated ring;
and additional double bonds may be present between C atoms 4 and 5; 5 and 6, and/or if appropriate to derivatize them in a manner which is known per se;
whereby alcohols are oxidized, or are acylated, sulpamoylated, sulphated, phosphorylated, alkylated or silylated using an acid derivative, an alkylation agent, or a silylating agent in the presence of a base and a suitable solvent;
keto groups, and in the case of the Birch reduction the aromatic A ring as well, are reduced in known manner or are protected by ketalization or thioketalization;
double bonds are displaced by treatment with bases or acids, enol ethers are cleaved by acids and double bonds are introduced by eliminating water from alcohols;
other double bonds in the vicinity of the aromatic A ring are introduced in a known manner by anodic oxidation, or additional double bonds are introduced by bromiding/dehydrobromiding;
aromatic ethers are cleaved in a known manner with boron tribromide or ethyl mercaptan and potassium ter-butylate;
the 17-keto function is converted in a known manner with Li/acetontrile or trimethylsulphonium iodide and subsequent treatment with a nucleophile to form compounds containing a 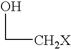
(X nucleophile);
wherein the order of reaction steps in the derivatization and the number of reaction steps may be changed or adjusted.

10. A method for the production of 8α,14β-dialkyl-18-norsteroids with general formula I according to claim 2, characterized in that the Weichert ketone (II) is pre-treated in an ether such as dimethoxyethane or tetrahydrofuran with a base such as sodium hydride or potassium tert-butylate and allowed to react with an alkylation synthone (III) such as 2(3-alkyloxyphenyl)-tosylate or -bromide and is transformed, after further action with a base using an alkylation agent such as methyl, ethyl or methoxymethyl iodide, into 17β-tert-butoxy-8α-alkyl-9-oxo-9,10-seco-gona-1,3,5(10),14-tetraene (IV): 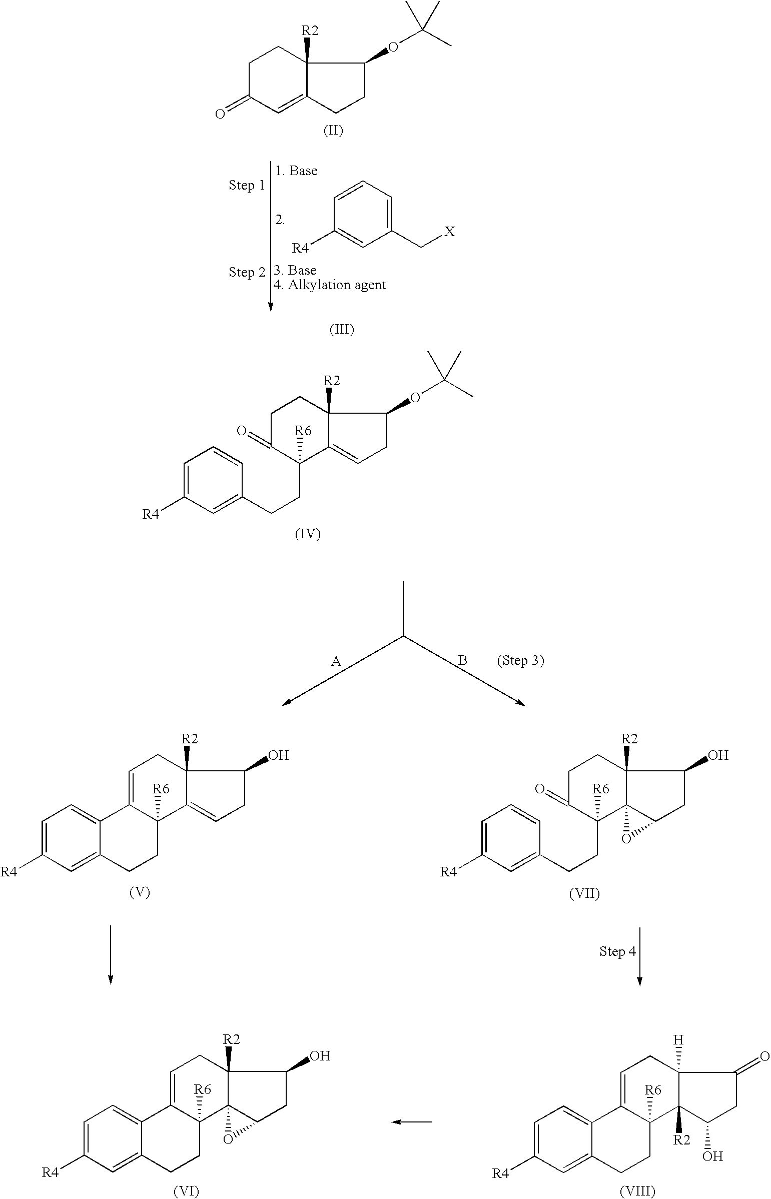
in which
R₂=methyl or ethyl;
R₄=alkyloxy (alkyl containing 1 to 12 C atoms), alkyloxymethyloxy (alkyl containing 1 to 3 C atoms);
R₆=methyl, ethyl or alkyloxymethyloxy (alkyl containing 1 to 3 C atoms);
X=tosyloxy, bromide, chloride or iodide;
and this is isolated either via route A:
transformation by aldol condensation using an organic acid such as p-toluenesulphonic acid or a Lewis acid such as boron trifluoride diethyl ether complex in an aprotic solvent into 17β-hydroxy-8α-alkyl-gona-1,3,5(10),9(11),14-pentaene with general formula (V), in which R₄ and R₂ have the meanings defined above, the latter being reduced, by selective epoxidation using peracids such as m-chloroperbenzoic acid or by Sharpless epoxidation using vanadylacetyl acetonate and tert-butyl hydroperoxide in any solvent such as toluene, to the 14α,15α-epoxide with general formula (VI), this latter being transformed, by treatment with a Lewis acid such as boron trifluoride diethyl ether complex in a suitable anhydrous solvent such as toluene, into 8α,14β-dialkyl-15α-hydroxy-17-oxo-gona-1,3,5(10),9(11)-tetraene (I or VIII with the meanings defined above for R₂ and R₄);
or via route B:
reduction to the seco-14α,15α-epoxide (VII) by selective epoxidation using peracids, which, by means of an organic acid or Lewis acid such as boron trifluoride diethyl ether complex in a suitable anhydrous solvent such as toluene, is transformed into 8α,14β-dialkyl-5α-hydroxy-17-oxo-gona-1,3,5(10),9(111)-tetraene (I or VIII with the meanings defined above for R₂ and R₄);
and/or this may if appropriate be transformed or derivatized in a manner which is known per se;
whereby alcohols are oxidized, or are acylated, sulpamoylated, sulphated, phosphorylated, alkylated or silylated using an acid derivative, for example chlorinated hydrocarbon chloroalkyl ester, an alkylation agent, or a silylating agent in the presence of a base and a suitable solvent;
keto groups are protected by ketalization or thioketalization, and these as well as double bonds, in the case of the Birch reduction the aromatic A ring as well, are reduced in known manner with complex hydrides or by catalytic hydrogenation;
double bonds are isomerized in known manner by treatment with bases or acids or are introduced by eliminating water from alcohols and enol ethers are cleaved by acids;
other double bonds in the vicinity of the aromatic A ring are introduced in a known manner by anodic oxidation, or additional double bonds are introduced by bromiding/dehydrobromiding;
aromatic and aliphatic ethers are cleaved in a known manner with boron tribromide or ethyl mercaptan and potassium ter-butylate or trimethylsilyl iodide;
the 17-keto function is converted in a known manner with Li/acetontrile or trimethylsulphonium iodide and subsequent treatment with a nucleophile to form compounds containing a 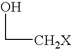
group
(X=nucleophile);
wherein the order of reaction successive steps in the derivatization steps and the number of reaction steps may be changed or adjusted.

11. A method for the production of spiro[cyclopentanoperhydronaphthalene]-3,1′-pentanes (-hexanes) with general formula I according to claim 3, characterized in that 17α-hydroxy-14β,15β-epoxysteroids with general formula II: 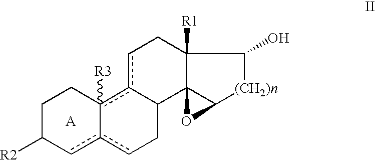
in which
R₁=methyl or ethyl;
R₂=OH, O, alkoxy (alkyl containing 1 to 12 C atoms), alkyloxymethyloxy (alkyl containing 1 to 3 C atoms), alkanoyloxy (alkyl containing 1 to 12 C atoms), aralkanoyloxy (alkyl containing 1 to 4 C atoms), aralkyloxy (alkyl containing 1 to 3 C atoms), aralkanoyloxy (alkyl containing 1 to 4 C atoms);
R₃=methyl or H in the case in which A does not represent an aromatic ring and there is no double bond between C atoms 5 and 10 and 9 and 10;
n=1 or 2;
A=an aromatic, partially or completely hydrogenated ring;
and additional double bonds may be present between C atoms 4 and 5, 5 and 6, 5 and 10, 9 and 10 and/or 9 and 11;
in an anhydrous aprotic solvent such as toluene, are converted in the presence of an acid such as p-toluenesulphonic acid or a Lewis acid such as boron trifluoride, to isolate spiropentanes (hexanes) with general formula Ia: 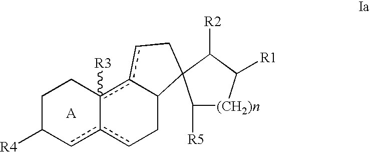
in which
R₁=methyl or ethyl;
R₂=OH, O, alkoxy (alkyl containing 1 to 12 C atoms), alkyloxymethyloxy (alkyl containing 1 to 3 C atoms), alkanoyloxy (alkyl containing 1 to 12 C atoms), aralkanoyloxy (alkyl containing 1 to 4 C atoms), aralkyloxy (alkyl containing 1 to 3 C atoms), aralkanoyloxy (alkyl containing 1 to 4 C atoms);
R₃=methyl or H in the case in which A does not represent an aromatic ring and there is no double bond between C atoms 5 and 10 and 9 and 10;
n=1 or 2;
A=an aromatic or partially hydrogenated ring;
and additional double bonds may be present between C atoms: 4 and 5, 5 and 6, 5 and 10, 9 and 10 and/or 9 and 11;
and/or they may, if appropriate, be transformed in a manner which is known per se;
whereby alcohols are oxidized, or are acylated, sulpamoylated, sulphated, phosphorylated, alkylated or silylated using an acid derivative, an alkylation agent, or a silylating agent in the presence of a base and a suitable solvent;
keto groups, and in the case of the Birch reduction the aromatic A ring as well, are reduced in known manner with complex hydrides or by catalytic hydrogenation or are protected by ketalization or thioketalization;
double bonds are isomerized by treatment with bases or acids, enol ethers are cleaved by acids and double bonds are introduced by eliminating water from alcohols;
other double bonds in the vicinity of the aromatic A ring are introduced in a known manner by anodic oxidation, or additional double bonds are introduced by bromiding/dehydrobromiding;
aromatic and aliphatic ethers are cleaved in a known manner with boron tribromide or ethyl mercaptan and potassium ter-butylate or trimethylsilyl iodide;
the 17-keto function is converted in a known manner with Li/acetontrile or trimethylsulphonium iodide and subsequent treatment with a nucleophile to form compounds containing a 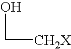
(X=nucleophile);
wherein the order of successive reaction steps in the derivatization steps and the number of reaction steps may be changed or adjusted.

12. A method for the production of 14β-fluoro, 15β,17β-dihydroxysteroids with general formula I according to claim 4, characterized in that 17β-hydroxy-14β,15β-epoxysteroids with general formula II: 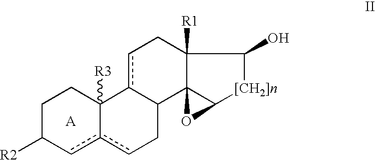
in which
R=methyl or ethyl;
R₂=O, OH, O-alkyl containing 1 to 12 C atoms, alkyloxymethyloxy (alkyl containing 1 to 3 C atoms), alkanoyloxy containing 1 to 12 C atoms, aralkanoyloxy (alkyl containing 1 to 4 C atoms) or aralkyloxy (alkyl containing 1 to 4 C atoms);
R₃=methyl or H in the case in which A does not represent an aromatic ring;
n=1 or 2;
A=an aromatic ring or a partially hydrogenated ring;
and additional double bonds may be present between C atoms 4 and 5; 5 and 6 and 9 and 11;
is converted by means of BF₃*Et₂O in an anhydrous aprotic solvent such as toluene and the resulting 14β-fluoro, 15β-hydroxysteroids with general formula Ia: 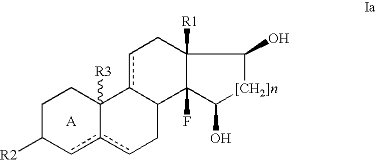
in which
R₁=methyl or ethyl;
R₂=O, OH, O-alkyl (alkyl containing 1 to 12 C atoms), alkyloxymethyloxy (alkyl containing 1 to 3 C atoms), alkanoyloxy (alkyl containing 1 to 12 C atoms), aralkanoyloxy (alkyl containing 1 to 4 C atoms) or aralkyloxy (alkyl containing 1 to 4 C atoms);
R₃=methyl or H in the case in which A does not represent an aromatic ring;
n=1 or 2;
A=an aromatic ring or a partially hydrogenated ring;
and additional double bonds may be present between C atoms: 4 and 5; 5 and 6 and 9 and 11, is isolated;
and/or it is derivatized or transformed in known manner;
whereby alcohols are acylated, sulpamylated, sulphated, phosphorylated or alkylated using an acid derivative or an alkylation agent in the presence of a proton-abstracting base and a suitable solvent;
the aromatic A ring is reduced in known manner using the Birch reduction and converted into 3-keto-Δ⁴- or 3-keto-5(10) compounds by acid treatment;
double bonds in the vicinity of the aromatic A ring are introduced in a known manner by anodic oxidation, or additional double bonds are introduced by bromiding/dehydrobromiding from 3-keto-5(10 compounds;
keto groups are reduced in known manner using complex hydrides or by catalytic hydrogenation;
aromatic and aliphatic ethers are cleaved in a known manner with boron tribromide, ethyl mercaptan and potassium ter-butylate or trimethylsilyl iodide;
wherein the order of successive reaction steps in the derivatization steps and the number of reaction steps may be changed or adjusted.

13. Pharmaceutical compositions containing at least one 14β-alkyl-18-norsteroid according to claim 1, a 8α,14β-dialkyl-18-norsteroid, a spiro[cyclopentano-perhydronaphthalene]-3,1′-pentane (-hexane) or a 14β-fluoro-15β,17β-dihydroxysteroid, optionally with pharmaceutically acceptable excipients, carriers and/or stabilizers.

14. Pharmaceutical preparations according to claim 13, characterized in that administration is by systemic or topical, oral, subcutaneous or dermal means or by injection.
